(19) United States
(12) Patent Application Publication
(10) Pub. No.: US 2015/0274972 A1
(43) Pub. Date: Oct. 1, 2015

(54) ENHANCED WATER AND TRANSFER RESISTANT FILM FORMING

(71) Applicant: ALZO INTERNATIONAL, INC., Sayreville, NJ (US)

(72) Inventors: Juan R. Mateu, Oak Ridge, NJ (US); Albert A. Zofchak, Holmdel, NJ (US); Michael R. Mosquera, Forked River, NJ (US); Michael R. Batko, Parlin, NJ (US); Nagi Awad, Franklin Lakes, NJ (US); Brianna Bicho, East Brunswick, NJ (US)

(73) Assignee: ALZO INTERNATIONAL, INC., Sayreville, NJ (US)

(21) Appl. No.: 14/434,180

(22) PCT Filed: Oct. 8, 2013
(86) PCT No.: PCT/US13/63845

§ 371 Date: Apr. 8, 2015

Related U.S. Application Data
(60) Provisional application No. 61/713,102, filed on Oct. 12, 2012.
(60) Provisional application No. 61/788,603, filed on Mar. 15, 2013.

(51) Int. Cl. C08L 83/06; C08J 3/18; A61Q 1/10; A61K 8/891; A61Q 1/06; A61Q 19/00; C08L 75/08; A61K 8/87

(52) U.S. Cl. CPC C08L 83/06; C08L 75/08; C08J 3/18; A61K 8/87; A61K 8/891; A61Q 1/06; A61Q 19/00; A61Q 1/10; C08L 2203/16; C08L 2203/02; C08J 2383/06; C08J 2375/08; C08J 2483/06; C08J 2475/08; A61K 2800/10

(57) ABSTRACT

The present invention relates to novel films prepared from a water dispersible thermoplastic resin and one or more silicone elastomers, which are optionally chemically modified to contain at least one internal plasticizer optionally including an emulsifier, which produce an integral film when placed on a substrate which exhibits unexpectedly exceptional characteristics of enhanced water resistance, wear, adhesion and transfer resistance characteristics as defined herein. These compositions may be further combined with components typically used in personal care products to produce personal care compositions, especially including lipsticks and color cosmetics, among numerous others.

### RELATED APPLICATIONS AND CLAIM OF PRIORITY

This application claims priority from U.S. provisional application Ser. No. 61/713,102, filed Oct. 12, 2012 entitled “Enhanced Water and Transfer Resistant Film Forming” and U.S. provisional application Ser. No. 61/788,603, filed Mar. 15, 2013, also entitled “Enhanced Water and Transfer Resistant Film Forming”, both of which applications are incorporated by reference in their entirety herein.

### FIELD OF THE INVENTION

The present invention relates to novel films prepared from one or more water dispersible thermoplastic resins and one or more thermoplastic elastomers, including silicone elastomers, optionally including an emulsifier, which produce an integral film when placed on a substrate which exhibits unexpectedly exceptional characteristics of enhanced water resistance, wear, adhesion and transfer resistance characteristics as defined herein. Said film can be formulated to yield water soluble ingredients and oil soluble ingredients delivered onto the applied substrate. These compositions may be further combined with components typically used in personal care products to produce personal care compositions, especially including lipsticks, lipglosses and color cosmetics, among numerous others.

### BACKGROUND AND DISCUSSION OF THE PRESENT INVENTION

The present invention relates to the use of elastomers, often silicone elastomers as plasticizers in a water based or water dispersed thermoplastic adhesive. In the present invention, plasticizers impart flexibility in a film comprising a thermoplastic resin or adhesive by creating spaces between the molecules of the adhesive polymer. These spaces can reduce the glass transition of the polymer which softens the polymer. Softening of the polymer often reduces the viscosity of the adhesive. Reducing the viscosity of a polymer often aids in the diffusion of the polymer onto and into a substrate. Diffusion of the adhesive onto the substrate creates better adhesion due to its increased contact or wetting of the surface and often reduces voids in the adhesive/substrate interface, thus increasing the adhesive to substrate contact points.

A mono-molecular adhesive produces the strongest bond. This mono-molecular adhesive must have sufficient adhesion and be cohesive. Cohesiveness of the polymer means that as the water and or solvent evaporates, the molecules in the adhesive will come into contact and join together to form a continuous film. Plasticizers very often cause a reduction in the cohesiveness of the film since the plasticizer will cause separation of the adhesive molecules, resulting in the diminution of the favorable characteristics of the film. A continuous film often denotes the lack or minimization of free spaces or voids in the film. Any voids or micro flaws in the film reduces adhesion and therefore reduces contact with the substrate to which the film is applied to, resulting in weakness in the film. These weaknesses are the points of contact where the film's bond and adhesion to the substrate, will start to fail.

Film failure can be defined as a loss of adhesion, reduction in water resistance, and a transfer of any part of the film to a second surface upon contact of the film with that second surface. Transfer resistance is typically associated with products which are colored. A transfer of color denotes a failure of the film and leads to a decrease in wear properties. These are considered undesirable characteristics of a film Another aspect of film failure can be caused by over plasticizing a thermoplastic film. Over-plasticity will cause the film former to be tacky or sticky. This in turn may result in a film that never sets fast enough or never sets.

There are many things that can contribute to the formation of micro flaws in an adhesive film, including the composition of the adhesive, the viscosity of the adhesive, the condition of the substrate, the evaporation rate of solvent in which the adhesive is dispersed, diffusion of the adhesive onto the substrate, and the inclusion of other ingredients which contribute to the formation of micro flaws in the film resulting in weakness and lack of durability. Any ingredient that is added to and becomes part of the adhesive film other than the adhesive itself often reduces cohesion and adhesion. Homogeneity of the composition is critical in delivering an optimum film. Specifically, in the current invention, homogeneity of the thermoplastic and the elastomer (preferably, a thermoset elastomer) will lead to an optimal film being formed.

In traditional polymer production, a plasticizer can be internalized by adding the plasticizer to the monomers during the production of a thermoplastic polymer or grafting the plasticizer after formation of the thermoplastic polymer. Alternatively, as in certain preferred embodiments of the present invention, the plasticizer may be grafted onto the silicone elastomer (preferably, a thermoset silicone elastomer) as a secondary internal plasticizer to provide an additional internal plasticizer moiety to the thermoset silicone elastomer. As discussed above, it is also noted that an internal plasticizer may also be polymerized into thermoplastic resin. In this manner, the plasticizer becomes part of the molecule and instills favorable characteristics to the final film while maintaining integrity and durability of the film as a consequence of the copolymerization or chemical bonding (grafting) of the plasticizer to the silicone elastomer and/or to the thermoplastic resin. Pursuant to the present invention, a plasticizer may also be external, but in such manner, is typically understood to be less desirable than a copolymerized plasticizer (i.e., copolymerized with or becoming integral to the polymer which is formed by monomer polymerization). In contrast, an external plasticizer added to a thermoplastic polymer is a plasticizer which is in physical interaction with the thermoplastic adhesive molecule. It may be theorized that the main role of an external plasticizer may be to lower the glass transition temperature of the polymer. Internal plasticizers can also lower glass transition. Internal plasticizers are limited due to stoichiometric constraints. Limited reactive sites limit the amount of internal plasticizers. This is often the case with thermoplastic elastomers. In the case of cross-linked thermoset elastomers the reactants used to create such elastomers contain more reactive sites and can lead to greater accommodation and higher molar concentration and multiplicity of internal plasticizers. Levels of internal plasticizers as well as multiple and varied internal plasticizers are possible.

Internal plasticizers for use in the present invention include monomeric and polymeric esters, ethers, alcohols, waxes, oils, hydrocarbon polymers, etc. which can be polyermized or reacted into the thermoplastic resin and/or elastomer (especially a thermoset silicone elastomer) in order to form a bond which is chemically integral with the polymer. The majority of plasticizers used are esters, but various ethers, alcohols, waxes, oils and hydroarbons may be modified to be reactive with thermoplastic and/or elastomer components and/or polymers. Exemplary internal plasticizers include any plasticizer which is modified to contain a group which is reactive with other monomers to form a thermoplastic polymer and/or an elastomer polymer or with reactive groups (e.g vinyl, alkenyl, hydroxyl, amine, carboxyl, Si—H, etc.) on a thermoplastic polymer or elastomer polymer precursor which can be reacted to form a final thermoplastic polymer or elastomer polymer for use in compositions according to the present invention.

Preferred plasticizers for incorporation as internal plasticizers into thermoplastic or preferably crosslinked silicone elastomers pursuant to the present invention include, for example, cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, Di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂-C₁₃ alkyl lactate, C₁₂-C₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂-C₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxstearate or a mixture thereof, among others.

External plasticizers can be sub-categorized into two types. The first type is referred to as a primary external plasticizer and its role is to reduce the glass transition of the thermoplastic resin. The second type is called a secondary external plasticizer and it is used as an aid to the primary external plasticizer. If the plasticizer has a lower glass transition than the thermoplastic molecule glass transition may be reduced, depending upon the interaction of the plasticizer with the thermoplastic resin. The internalization of the plasticizer can reduce the unfavorable chracteristics produced by the addition of an external plasticizer. It can reduce/increase the brittleness of a film or it could add tack, and or shine to a film. The one clear advantage of an internal plasticizer is that it eliminates the migration of the plasticizer from the film because it is chemically bonded to the film. Shine or tack produced by an external plasticizer can overtime migrate from the film whereas an internal plasticizer is bonded to the elastomers comprising the film and migration of this internalized plasticizer is not possible.

Generally, the inclusion of a plasticizer creates micro flaws in a monomolecular film, which instead of being characterized as a mono molecular film, a continuous film, becomes characterized unfavorably as a multi molecular or a discontinuous film. While the choice of plasticizer can minimize the reduction of cohesion/adhesion of the film, generally, the inclusion of a plasticizer results in largely diminished film characteristics. Ideally, the most favorable outcome would be a plasticizer that creates a homogenous film formation and contributes to film formation. Typical external plasticizers can be selected from the following chemical compounds: esters, ethers, alcohols, waxes, oils, hydrocarbon polymers, etc. The majority of plasticizers used are esters.

The use of plasticizers in a thermoplastic resin adhesive will not contribute to the strength of the film formed. Strength of film may be indicated by such factors as the water resistance and transfer resistance qualities that lead to a long lasting film formation. Tensile strength of the film can also be weakened by the use of plasticizers. This can be a favorable outcome in certain instances. Making a rigid crystalline film into a flexible film can be a favorable and desired attribute. Flexibility can lead to durability of the film as well as a film that conforms to the substrate and can move with the substrate. The addition of plasticizers only diminishes the quality of the film, sometimes substantially. However, a plasticizer which is multifunctional may be used to eliminate or minimize the negative impact the addition of a mono functional plasticizer.

The present invention utilizes silicone elastomers to plasticize thermoplastic resin adhesives. Silicone elastomers are used in cosmetic compositions for various functions. Elastomers and specifically silicone elastomers are not typically used as plasticizers for a water based or water dispersed thermoplastic resin/adhesive. Silicone polymers are well known materials in the cosmetic and personal care industries. These materials are composed of repeating dimethylsiloxanes units which may be terminated in various ways. These terminations, in large measure, determine their properties (i.e. hydrophillic) and their ability to react with other materials. In addition to the terminal groups, the polydimethylsiloxanes themselves can have widely varying molecular weights and may be linear, branched or cross linked structure. Each of these variations will produce widely varying properties and uses. Some of the silicone polymers that are more useful in cosmetic and personal care products are the silicone elastomers. These are highly cross-linked, non-vulcanized silicone polymers that have physical properties that resemble those of rubber—in that they deform and stretch when force is applied, they bounce when dropped to the floor and they exhibit an elastic memory, i.e. exerting a force to return to their original shape once they are deformed. When diluted (or dispersed) in a solvent (or liquid) these elastomers find use as film forming materials. Further, when used in cosmetics or personal care products, the elastomer solutions or dispersions produce a very smooth, non-oily, dry feeling lubricity on skin and hair. This effect is especially appreciated in make-up products that contain pigments and because the product application is greatly improved and because the elastomer film can reduce the rubbing off of the pigments once the product is applied and has dried. The silicone elastomers of particular interest are those described in U.S. Pat. No. 6,936,686 which is directed to the preparation and use of silicone elastomers that are cross linked as a solution in either a low viscosity silicone oil, a hydrocarbon oil, a cyclomethicone or mixtures thereof. These polymers may also be prepared in cosmetic esters, as described in international application publication WO2009/054931, Apr. 30, 2009.

Cross linked silicones are described as having a cage-like or scaffold structure. This cross linking is what gives this ingredient its solvent gelling ability. Compatible solvents occupy the spaces formed by the cross linked structure. This results in a swelling of the 3-dimensional cross linked silicone structure. Silicone elastomers tend to be very cohesive. The less solvent the more cohesive the elastomer molecules are. This cohesiveness is evident in the ability of this type of material to form films on the skin. These films are known to resist transfer. The cohesion of the silicone elastomer holds the film and ingredients included in a composition on the skin or hair.

Fumed silica has been used to reinforce silicone elastomers. The silica when mixed with the elastomer will fill the spaces in the scaffold like structure. The silica displaces the solvent or gels the solvent that is occupying these spaces. This increases the cohesion of the molecule. Increasing cohesion of the thermoset elastomer increases viscosity. This increase in viscosity can help maintain stability after the cross linked silicone thermoset elastomer is emulsified.

Silicone elastomers are known to create films on the skin. This film however, due to the cross linked structure of the silicone elastomer, will produce a discontinuous film. Once this film is applied to the substrate it can never be removed in one or several pieces. The removal of the film is accomplished by wiping away the film. Water based thermoplastic resins form continuous films. This film can be removed by peeling the film in parts or its entirety. In essence, a thermoset silicone elastomer and thermoplastic resin exhibit characteristics which are quite different and suggest that such a combination would have mediocre at best, if not poor, combined film characteristics. This is because the attributes of each of these materials would work at cross purposes, rather than complimentary purposes. Mixing of the two materials would not be expected to result in a homogenous mixing, which in turn, would produce a film with discrete portions of the film being either thermost or thermoplastic.

The novel use of silicone elastomers, preferably, cross-linked silicone thermoset elastomers as a plasticizer of the thermoplastic creates better film attributes than the use of conventional plasticizers as previously described.

The affinity of the thermoplastic polymer and the thermoset elastomer enable the thermoset elastomer to function as a plasticizer during the mixing of the two materials, thermoset and thermoplastic. Once mixed together, a homogeneous emulsion is formed. Typical cross linked silicone thermoset elastomers are extremely cohesive. Reduction of this cohesiveness can be induced by the introduction of polar ingredients. Total collapse of the gel can occur if the ingredients are too polar or cationic. Water borne thermoplastic are by their nature polar and or ionic. The mixing of a water borne thermoplastic elastomer with a cross linked silicone elastomer reduces the surface tension of the preferred silicone crosslinked thermoset elastomer and allows for the plasticizing of the water borne thermoplastic. Plasticity can relate to the “workability” of a thermoplastic and not only the plasticity of the set plastic or set plastic film. “Workability” can be defined as the ability of the mixed composite to be mixed or worked. The induction of polarity cause the preferred silicone cross linked thermoset elastomers gel structure surface tension to reduce. This reduction allows for the flow of the water borne thermoplastic into the cross linked silicones structure. Once the water borne thermoplastic is mixed into the cross linked silicones structure it acts as an intermolecular spacer keeping the water borne thermoplastic from coming together and physically bonding as it could if it was a monomolecular film. If the silicone cross linked thermoset elastomer is dispersed in a volatile solvent such as isododecane. The mixture of silicone cross linked thermoset elastomer and water borne thermoplastic when applied to a substrate will create a dry film in which the water borne thermoplastic is kept apart or plasticized by the silicone cross linked thermoset elastomer. The film is also reinforced and or enhanced by the presence of the two polymers (thermoplastic and thermoset) creating said film. It's not a thermoplastic film and its not a thermoset film. It's a combination of both and the film shares the physical attributes of the two.

If the silicone cross linked thermoset elastomer is dispersed in a non-volatile solvent such as isononyl isononanoate, the mixture of silicone cross linked thermoset elastomer and water borne thermoplastic when applied to a substrate will create an oily film in which the water borne thermoplastic is kept apart or plasticized by the silicone cross linked thermoset elastomer and the isononyl isononanoate. The film is also reinforced and or enhanced by the presence of the two elastomers creating said film. It's not a thermoplastic film and its not a thermoset film. It has attributes of both and the film shares the physical characteristics of the two plus the contribution from a quantity of isononyl isononanoate. This quantity of isononyl isononanoate can be as high as 70%. This addition of non-volatile solvents are considered plasticizers along with the silicone cross linked thermoset elastomer. All volatiles and or non-volatile solvents, oils, esters, and silicone oils will behave in the same manner. Prior to the present invention, plasticizing of a thermoplastic with high levels of non-volatile solvent would create a very tacky film that would not dry or set for a long time or not at all.

In the current invention, the present inventors describe the plasticizing of the thermoplastic. One definition is that the plasticizer is internally or chemically bonded to the thermoplastic. The other is that the plasticizer is external and physically bonded. The same can be said of the cross linked silicone thermoset. Cross linked silicone thermoset can be internally plasticized and externally plasticized. When the two, thermoplastic and thermoset are emulsified, the thermoplastics physical nature, as a liquid dispersion, allows the thermoplastic to mix with the thermoset, a solid. The thermoset acts/functions as a molecular spacer and keeps the thermoplastic from achieving its highest level of physical bonding. In essence, the thermoset functions to plasticize the thermoplastic. There can also be a description of the thermoset not being allowed to cohere/physically bond fully with itself due to the presence of the thermoplastic in the film.

The addition of non-volatile solvents to disperse the cross linked thermoset elastomer can also have a plasticizing affect on the film. Essentially, it is the plasticizing characteristic of the elastomer, which may be supplemented with a further external or internal plasticizer (in either the elastomer or the thermoplastic) which provides favorable cohesion of the two materials to produce an unexpectedly superior film exhibiting properties which is an amalgam of the two materials.

Amines interact with water to form ammonium hydroxide. Primary, secondary and tertiary amines all are water soluble. Primary amines are the most water soluble while tertiary amines are less water soluble. The water solubility or hydrophilicity of a tertiary amine is reduced by its hydrophobic moiety. This can aid in dispersing pigments in non-ionic emulsions. Its cationic nature makes a silicone elastomer which is typically non-polar into a polar molecule. A silicone elastomer which has amine functionality grafted onto its backbone can have a neutralizing effect on pH as well as reduce the surface tension of the cross linked silicone elastomer.

A silicone elastomer that has film forming properties, emulsifying properties, enhanced solubility of both hydrophilic and lipophillic properties and has a cationic charge is an example of a multifunctional silicone elastomer. Surprisingly, these attributes are ideal for blending water based thermoplastic resins and achieving a compositional result that is far superior to past attempts at creating long lasting, water and transfer resistant films. The emulsifying potency of either the thermoset elastomer or the thermoplastic can impact the films water resistance. Additional external emulsifier will also impact the films water reistance. The multifunctional elastomer described above can emulsify, aid in pigment dispersion, exhibit film forming properties, increased compatibility with hydrocarbons and silicone type ingredients, and can play a role in controlling the pH. Silicone elastomers which are used in the present invention function as plasticizers for the thermoplastic resin. External plastizers may be added to the silicone elastomer and/or the thermoplastic resin to enhance the plasticizer characteristics of the final film. In certain preferred embodiments according to the present invention, the elastomer (in preferred embodiments a thermoset silicone elastomer) is further functionalized to incorporate internal plasticizers by copolymerization with monomers to produce internally plasticized silicone elastomers and/or grafted onto preformed silicone elastomers which contain functional groups to which the plasticizer molecules may be grafted. These are described in greater detail hereinbelow.

Typical silicone elastomers that have not been hydrophilically modified or do not contain amine moieties can plasticize water based thermoplastic resins. These same silicone elastomers may be further modified chemically to incorporate a secondary internal plasticizer to provide further plasticizing characteristics as otherwise described herein. By chemically modifying the silicone elastomers (typically thermoset silicone elastomers) used in the present invention by copolymerization with monomers to produce the silicone elastomers, or alternatively, by grafting plasticizer compounds onto the silicone elastomer as otherwise described herein, a stable secondary plasticizing component can be included in silicone elastomers and final compositions for the benefit that an additional plasticizing component may provide to the final compostions and films produced therefrom, including softness of the film with increased integrity, strength and durability by chemically bonding the secondary plasticizer to the silicone elastomer.

A multifunctional silicone elastomer as previously defined would be soluble in hydrocarbon based ingredients, soluble in silicone based ingredients, hydrophilic, and carries a cationic charge. A silicone elastomer combined with an emulsifier would enable water based materials to be used in emulsions. Both the elastomer and the emulsifier would have to be soluble and or compatible with thermoplastic resins. Compatibility would ensure that a homogenous film formation would be made. This is critical in maintaining proper adhesion and just as important cohesion would be maintained. A cationic ingredient such as amodimethicone can be used to aid in dispersing pigments. The cationic ingredient can also aid in pH control. A secondary plasticizer component integral (i.e., chemically bonded) to the polymer may add softness to the final film characteristics with an added benefit of increased integrity, strength and durability compared to compositions which add external plasticizers to the final compositions from which films are made. For example, the addition of an internal plasticizer or multiple internal plasticiczers on the backbone of a cross linked silicone elastomer or thermoplastic can produce different physical as well as sensorial characteristics. Addition of internal plasticizers can change physical attributes such as solubility parameters, polymer content (% polymer in solvent), refractive index, adhesion, cohesion etc. Addition of internal plasticizers containing alkene moiety will reduce cross linking percentage of the silicone elastomer (cross linked silicone elastomers usually contain bis-vinyl or di-vinyl moieties in the polymer precursors). If this monomer is replaced with an alkene moiety there will be less cross linking as the alkene will react as a pendant group. This reduction in crosslinking allows for extensive manipulation of the silicone cross linked elastomer. Sensorial attributes are related to the feel and look of the cross linked silicone elastomers when applied on the substrate. Bis-vinyl hydrocarbons can replace di-vinyl silicones creating a harder or “Glassy” film.

In addition, cross linked silicone elastomer containing a higher hydrocarbon moiety than one made with a di-vinyl silicone are possible.

Water based film formers are used to create long lasting, transfer resistant, and water resistant films. Examples of these resin are as follows, Poly (vinyl acetate), Poly (vinyl alcohol), Poly (vinyl chloride) and Copolymers, Polyolefins and Copolymers, Polyesters, Polyamides, Acrylic Acid Copolymers, Methacrylic acid, Acrylamides, Methacrylates and Acrylates, water dispersible Polyurethanes, among others. These water based film formers are typically used as adhesives. When trying to bond one substrate to another these film formers are used. This invention will relate to the use of these materials in a composition that is applied on hair, nails, and on the skin.

Thermoplastic resin dispersion functions as a film former. It must also be able to form a composition that delivers typical cosmetic ingredients onto the skin or hair. The ideal composition would be viscous enough so that solids such as colorants would not settle to the bottom of the package. Most water dispersible thermoplastic resins tend to be low in viscosity. There are three ways to increase the viscosity of these materials. One is to use a water thickener. The second way is to use an emulsifier therefore creating an emulsion. The third way is to use both a water thickener and emulsifier. All three methods increase the water solubility of the Thermoplastic resin. Increased water solubility reduces the water resistance, transfer resistance, and adhesive qualities associated with Thermoplastic Resins.

U.S. Pat. Application Publication No. 20100260687, describes the use of an aqueous polyurethane dispersion in cosmetic compositions. This patent application describes the difficulties using these types of ingredients in the personal care industry. As previously described the use of water thickeners and emulsifiers are shown to be required when using water based thermoplastic Resins.

### BRIEF DESCRIPTION OF THE INVENTION

In one embodiment, the present invention is directed to novel polymer films exhibiting exceptional and unexpected film forming characteristics including uniformity, durability, flexibility, water resistance and transfer resistance. The compositions according to the present invention are particularly useful for providing compositions, in particular, topical pharmaceutical compositions and personal care products for use on keratinous surfaces, including the skin, hair and nails of a subject. The compositions according to the present invention are extremely adaptable, are generally applied, and allow for numerous final compositions to be provided for and further components to be added which make final formulations facile to make and generally applicable. The compositions are useful wherever a film is to be deposited onto an inanimate substrate such as plastic, woven and non-woven fibers, paper, wood, or rock. The compositions are useful wherever a film is to be deposited having favorable characteristics, especially including the topical delivery of active agents and providing for wound healing/wound care. The compositions according to the present invention are particularly useful as a liquid bandage, providing a durable thin film of exceptional biological properties. The compositions according to the present invention are also useful in providing personal care products, including color cosmetics, especially lipsticks, lipglosses, sunscreens, lotions, shampoos/conditioners, deodorants/anti-perspirants, hair care products, anti-aging formulations, and final compositions, depending on components, where superior film characteristics afford enhanced utility.

In a first embodiment, the present invention relates to a polymeric composition comprising at least one thermoplastic resin dispersed in an aqueous solvent wherein said thermoplastic resin optionally comprising an internal plasticizer chemically bonded thereto, preferably water or a mixture of water and alcohol; at least one elastomer (preferably, a silicone thermoset elastomer) optionally comprising an internal plasticizer chemically bonded thereto, in soluble, dispersible or gelled form in a solvent and an optional external emulsifier effective to emulsify said water and said solvent, wherein said thermoplastic resin comprises about 10% to about 80%, about 25% to about 75%, more often about 30% to about 65%, even more often about 40% to about 60% by weight of said dispersion (which includes solvent and thermoplastic resin); said elastomer comprises about 10% to about 100% (i.e., solvent may be excluded in certain instances and the thermoset resin is used neat as a gelled composition), about 10% to about 95%, about 10% to about 90%, about 10% to about 80%, preferably about 25% to about 75%, more often about 35% to about 65%, even more often about 40% to about 60% by weight of said elastomer and solvent, and wherein said emulsifier, when present, comprises about 0.01% to about 20% (often about 0.1% to about 10%, about 0.25% to about 7.5%) by weight of said elastomer, said solvent, said aqueous solvent and said thermoplastic resin in combination, said composition, when deposited onto a surface, dries (after evaporation of solvent and water) into a uniform, flexible, durable final film exhibiting water resistance, flexibility, durability and optionally, transfer resistance (when the final film also comprises a pigment, dye, oil and/or active), said thermoplastic resin comprising about 5% to about 95% by weight of said final film (often about 10% to about 80%, about 25% to about 75% by weight, often about 35% to about 65% by weight of said film, often about 45% to 55% by weight of said film or about 50% by weight of said film), said elastomer comprising about 0.5% to about 95% by weight of said film, (often about 1% to about 80%, often about 10% to about 75% by weight, often about 35% to about 65% by weight of said film, often about 45% to 55% by weight of said film or about 50% by weight of said film), said external emulsifier, when present, comprising about 0.025% to about 10% by weight of said film (often about 0.1% to about 7.5% by weight of said film).

The percentages noted above for thermoplastic polymer dispersions are percentages of “polymer dispersed in water”. Preferably, the polymer dispersions contain about 10-50% by weight thermoplastic polymer in about 50-90% water. Water based polyurethanes (as thermoplastic resins used in the present invention) often contain about 25-40%, more often about 30% polyurethane thermoplastic polymer. Polyurethane dispersions available from Alzo International, Inc. (Sayreville, N.J.) typically contain about 33% polyurethane thermoplastic polymer.

By way of example, in a preferred embodiment of the present invention, the ratios of components used in the invention are as follows: Waterborne polyurethane thermoplastic (about 33% polymer in water); Crosslinked silicone elastomer (preferably Nulastic series from Alzo International, Inc. (≈10% polymer in hydrocarbon, esters, silicone solvent) other brands may preferably contain up to 50% by weight thermoset silicone polymer in solvent. In certain preferred embodiments of the present invention, final compositions (which are deposited onto a surface to produce a final film) contain ≧10%≦90% waterborne polyurethane dispersion+≧10%≦75% cross linked silicone elastomer dispersion. Preferred transfer resistant compositions according to the present invention contain about 30-60% of the waterborne polyurethane dispersion and about 20-60% of the cross linked silicone elastomer dispersion. It is noted that in the above-described embodiments, the percentages of cross linked silicone elastomer dispersion can increase if the 10% polymer level is reduced by dilution. Alternatively, if a cross linked silicone elastomer containing >10% polymer is used, the percentages used in the formula can be reduced.

In an embodiment of the present invention, the thermoplastic resin is an aqueous solvent dispersible polymer selected from the group consisting of acrylonitrile butadiene styrene polymers, polyacrylic or poly(meth)acrylic resins, celluloid cellulose acetate, cyclic olefin copolymers, ethylene-vinyl acetate, ethylene vinyl alcohol, a fluoroplastic, acrylic/polyvinylchloride copolymer, liquid crystal polymer, polyacrylonitrile, polyoxymethylene, polyamide (nylon), polycarbonate, polyamide-imide, polyaryletherketone, polybutadiene, polybutadiene/styrene copolymers, polybutadiene/acrylic copolymers, polybutadiene/acrylamide copolymers, polybutylene, polybutylene terephthalate, polycaprolactone, polychlorotrifluoroethylene, polyethylene terephthalate, polyhydroxyalkanoates, polyketone, polyester, polyethylene (both low and high density), polyetheretherketone, polyetherketoneketone, polyaryletherketone, polyetherimide, polyethersulfone, chlorinated polyethylene, polyimide, polylactic acid, polymethylpentene, polyphenylene, polyphenylene oxide, polyphenylene sulfide, polyphthalamide, polypropylene, polystryrene, polysulfone, polytrimethylene terephthalate, polyurethane, polyvinyl acetate, polyvinyl chloride, polyvinylidene chloride and styrene-acrylonitrile, among others.

In embodiments of the present invention the elastomer is a thermoset elastomer (generally, a crosslinked elastomer), optionally (often preferably) comprising an internal plasticizer bonded thereto (either through copolymerization or by grafting onto the thermoset elastomer). In alternative embodiments, the elastomer is a thermoplastic elastomer. In embodiments, the elastomer is a silicone elastomer, often an elastomer as described in U.S. Pat. No. 6,939,686, which is directed to the preparation and use of silicone elastomers that are crosslinked (thermoset) as a solution in either, a low viscosity silicone oil, a hydrocarbon oil, esters, a cyclomethicone or mixtures thereof. U.S. Pat. No. 6,939,686 is incorporated in its entirety herein. In still other embodiments, the elastomer is a silicone crosslinked hydrocarbon elastomer, as generally described in international PCT application PCT/US2012/0843, filed Mar. 29, 2012, which is incorporated by reference in its entirety herein.

In embodiments, the compositions may optionally include an external emulsifier in a weight ratio ranging from about 0.01% to about 10-15% by weight of the final polymer composition, which includes both the thermoplastic resin, the elastomer, preferably a thermoset elastomer and the solvents which are used for the thermoplastic resin and the elastomer. The compositions may also optionally comprise an external plasticizer (i.e., a plasticizer which is included in admixture with the other components) in a weight ratio often ranging from about 0.01% to about 80%, often about 0.5% to about 60%, often about 0.75% to about 50%, about 1% to about 50% about 50% to about 80%, about by weight of the final polymer composition (which includes solvent). It is noted that the external plasticizer may be included in large amounts in the thermoset elastomer composition and small amounts, if at all, in the thermoplastic resin composition. Final films (after deposition onto a surface and evaporation of solvent) may contain upwards of about 60% by weight of an external plasticizer, often between about 0.05% to about 60%, about 0.25% to about 50%, about 0.5% to about 15%, about 1% to about 10%, about 0.75% to about 20%, about 1.5% to about 40%, about 30-60% by weight of an external plasticizer.

In embodiments, the thermoplastic resin and/or the elastomer may be self-emulsifying such that the external emulsifier may be eliminated or reduced in compositions according to the invention.

In certain embodiments, the present invention combines a silicone elastomer (non-vulcanized) with a water based thermoplastic resin. A film created by this compositional blend creates a continuous film. This film can be peeled or lifted off the substrate in one piece. The elastomer becomes an integral part of the thermoplastic resin, producing films of highly favorable characteristics. A cast film of a silicone elastomer and thermoplastic resin pursuant to the present invention will create a flexible, continuous and durable film. This exhibits not only a plasticizing effect but a reinforcement effect of the silicone elastomer, factors which could not be predicted. Quite surprisingly, the inclusion of the water based thermoplastic resin fills the voids of the cross linked elastomer and creates a hybrid film that exhibits enhanced qualities which work exceptionally well together. This allows for the current invention to be formulated as a cast film. This cast film can be used as a dermal patch that can deliver actives onto the surface of the skin. Pharmaceutical ingredients can be incorporated into this patch. The emulsion could also be applied onto the substrate and the patch can be created on the skin as well.

In certain embodiments, the present invention utilizes a silicone elastomer that has been hydrophilically functionalized, with a polyurethane and/or an allyl alcohol ethoxylate or other ethoxylate. U.S. Pat. No. 6,936,686 teaches the use of alkylene ethoxylate as a co-reactant to increase the hydrophilicity of the resulting elastomer. Such hydrophilic elastomers find great utility as water in oil (W/O) emulsifiers, as protectants and carriers for other water soluble ingredients and as fully functional silicone elastomers having the same expected properties previously described. Modification of the hydrophilic and hydrophobic content of either elastomer, crosslinked silicone elastomer and/or the water based thermoplastic could delay or speed up active delivery diffusion from the film to the substrate.

Compositions which relate to the formation of hydrophilic silicone elastomers from the admixture of a traditional hydrophobic silicone elastomer with hydrophilic polyurethane or the chemical reaction product of a silicone elastomer with an alkene containing hydrophilic polyurethane. These hydrophilic polyurethanes silicone elastomers provide additional solubility opportunities (because the polyurethane solubility characteristics can be widely varied i.e. increased hydrocarbon solubility), controllable hydrophilicity and variable deposition capacity (because the polyurethane can contain an amine or quaternary ammonium salt). An amine moiety helps in dispersing pigments in compositions that are typically nonionic. Amine functionality is typically known to have very good adhesion on surfaces that are anionic such as glass, plastic, and on skin and hair.

In certain embodiments of the present invention, a composition according to the present invention is comprised of the following:
(a) Thermoplastic film forming resin dispersed in water;
(b) Silicone elastomer in a volatile solvent or in non volatile solvent used as a plasticizer to plasticize the thermoplastic resin and produce films when the solvent evaporates (the silicone elastomer may also be used neat alone or in combination with an external plasticizer as otherwise disclosed herein;
(c) The thermoplastic resin and the silicone elastomer are optionally hydrophilically modified (self-emulsifying);
(d) The hydrophilically modified silicone elastomer can be further modified to include a polyurethane
(e) May contain: colorants, surface treated water soluble or oil soluble actives, sunscreen (organic, inorganic), emollients, clays, minerals, waxes, emulsifiers to provide personal care products;
(f) Can contain an external emulsifier or external plasticizer as otherwise described herein.

In certain embodiments, the present invention relates to a personal care composition comprising at least one thermoplastic resin dispersed in an aqueous solvent (thermoplastic resin dispersion), preferably water or a mixture of water and alcohol; at least one elastomer (preferably, at least one thermoset elastomer) in soluble, dispersible or gelled form in a solvent and an optional external emulsifier effective to emulsify said water and said solvent, wherein said thermoplastic resin comprises about 10% to about 80% (often, about 25% to about 75%, more often about 30% to about 65%, even more often about 40% to about 60%) by weight of said dispersion; said elastomer comprises about 10% to about 100%, about 10% to about 95%, about 10% to about 90%, about 10% to about 80% (preferably about 25% to about 75%, more often about 35% to about 65%, even more often about 40% to about 60%) by weight of said elastomer and solvent, and wherein said emulsifier, when present, comprises about 0.01% to about 20% (often about 0.1% to about 10%, about 0.25% to about 7.5%) by weight of said elastomer, said aqueous solvent and said solvent in combination, said composition further comprising at least one additional component (preferably two or more than two components) selected from the group consisting of water, an aqueous solvent (e.g. alcohol or other water compatible solvent), a non-aqueous solvent, emollients, humectants, oils (polar and non-polar), conditioning agents, emulsifiers, surfactants, thickeners, stiffening agents, medicaments, fragrances, preservatives, deodorant components, anti-perspirant compounds, skin protecting agents, pigments, sunscreens and mixtures thereof, among others, wherein said composition, when deposited onto a surface, dries (after evaporation of solvent and water) into a uniform, flexible, durable film exhibiting water resistance, flexibility, durability and optionally, transfer resistance when the film also comprises a pigment, dye, oil and/or active. In certain embodiments, the additional component(s) comprise about 0.05% to about 75% by weight of said personal care composition, about 0.1% to about 50%, about 0.25% to about 40%, about 0.5% to about 35%, about 0.75% to about 25%, about 1% to about 20%, about 2.5% to about 15%, about 2.5% to about 7.5%, about 5% to about 10% of said personal care composition, said composition, when deposited onto a surface, dries (after evaporation of solvent and water) into a uniform, flexible, durable film exhibiting water resistance, flexibility, durability and optionally, transfer resistance (when the film also comprises a pigment, dye, oil and/or active), said thermoplastic resin comprising about 5% to about 95% of said film (often about 25% to about 75% by weight, often about 35% to about 65% by weight of said film, often about 45% to 55% by weight of said film or about 50% by weight of said film), said elastomer comprising about 5% to about 95% by weight of said film, (often about 25% to about 75% by weight, often about 35% to about 65% by weight of said film, often about 45% to 55% by weight of said film or about 50% by weight of said film), said external emulsifier, when present, comprising about 0.025% to about 10% by weight of said film (often about 0.1% to about 7.5% by weight of said film), and said additional component(s) comprising about 0.1% to about 80% (often about 0.1% to about 25%, about 0.25% to about 15%, about 0.5% to about 10%, about 0.5% to about 5%, about 0.25% to about 2.5%) by weight of said film. It is noted that an external plasticizer may be included in the personal care compositions, in an amount up to about 60% by weight, often between about 0.05% to about 60%, about 0.25% to about 50%, about 0.5% to about 15%, about 1% to about 10%, about 0.75% to about 20%, about 1.5% to about 40%, about 30-60% by weight, depending upon the final attributes of the personal care composition.

Compositions according to the present invention may also be used to provide a liquid bandage which dries to a film covering the wound of a patient or subject, the composition comprising a polymeric composition as described above, optionally in combination with a bioactive or other additive selected from the group consisting of wound healing agents such as allantoin, aloe vera, antimicrobial agents, antiseptic agents, botanical extracts, colorants, fragrances, thickeners, vitamins (especially including vitamin E to reduce scarring) and mixtures thereof, among numerous others.

In still further embodiments, the present invention relates to pharmaceutical compositions which comprise a polymeric blend according to the present invention in combination with an effective amount of at least one bioactive agent adapted for topical or transdermal delivery of the bioactive agent to a patient or subject, the composition further comprising a pharmaceutically acceptable carrier, additive or excipient. In compositions which are formulated for transdermal delivery of at least one bioactive agent, the compositions may further include a penetration enhancing agent such as DMSO or other agent which facilitates the transdermal delivery of the agent through the skin of the patient of subject.

In still further embodiments, the present invention relates to a two part composition the composition comprising a part A mixture and a part B mixture,

the part A mixture comprising at least one thermoplastic resin dispersed in an aqueous solvent, preferably water or a mixture of water and alcohol and additional optional additives or bioactive agents;

the part B mixture comprising at least one elastomer (preferably, a single thermoset elastomer) in soluble, dispersible or gelled form in a solvent, said part A mixture and/or said part B mixture comprising an optional external emulsifier effective to emulsify said water and said solvent when said part A mixture and said part B mixture are combined, wherein said thermoplastic resin comprises about 10% to about 80% (often, about 25% to about 75%, more often about 30% to about 65%, even more often about 40% to about 60%) by weight of said part A mixture; said elastomer comprises about 10% to about 100%, about 10% to about 95% by weight, 10% to about 90% by weight, about 10% to about 80% by weight (preferably about 25% to about 75%, more often about 35% to about 65%, even more often about 40% to about 60%) by weight of said elastomer and solvent in said part B mixture and wherein said emulsifier, when present, comprises about 0.01% to about 10% (preferably about 0.1% to about 7.5%) by weight of said composition, said elastomer, said aqueous solvent and said solvent, said composition, when deposited onto a surface, dries (after evaporation of solvent and water) into a uniform, flexible, durable film exhibiting water resistance, flexibility, durability and optionally, transfer resistance (when the film also comprises a pigment, dye, oil and/or active), said thermoplastic resin comprising about 5% to about 95% of said film (often about 25% to about 75% by weight, often about 35% to about 65% by weight of said film, often about 45% to 55% by weight of said film or about 50% by weight of said film), said elastomer comprising about 5% to about 95% by weight of said film, (often about 25% to about 75% by weight, often about 35% to about 65% by weight of said film, often about 45% to 55% by weight of said film or about 50% by weight of said film), said external emulsifier, when present, comprising about 0.025% to about 10% by weight of said film (often about 0.1% to about 7.5% by weight of said film). In certain aspects, the part A and part B composition may comprise additional components in either the part A mixture or the part B mixture (depending upon compatibility) wherein said additional component(s) comprise about 0.1% to about 80% (often about 0.1% to about 25%, about 0.25% to about 15%, about 0.5% to about 10%, about 0.5% to about 5%, about 0.25% to about 2.5%) by weight of the final film. In addition, an external plasticizer may be included (preferably in the part B mixture comprising the elastomer) in a weight ratio often ranging from about 0.01% to about 80%, often about 0.5% to about 60%, often about 0.75% to about 50%, about 1% to about 50% about 50% to about 80% by weight of the final polymer composition (part A or Part B mixture—which generally includes solvent). It is noted that the external plasticizer may be included in large amounts in the thermoset elastomer composition (part B mixture, which may exclude solvent and use the elastomer neat without solvent) and small amounts, if at all, in the thermoplastic resin composition (part A). Final films (after deposition onto a surface and evaporation of solvent when parts A and B are deposited onto a surface and mixed) may contain upwards of about 60% by weight of an external plasticizer, often between about 0.05% to about 60%, about 0.25% to about 50%, about 0.5% to about 15%, about 1% to about 10%, about 0.75% to about 20%, about 1.5% to about 40%, about 30-60% by weight of an external plasticizer.

Compositions according to the invention may be presented as a unitary composition or as a two part, part A and part B composition.

Methods of applying these compositions onto a surface, especially including a keratinous surface (skin, hairs nails of a subject or patient) represent additional embodiments according to the present invention.

### BRIEF DESCRIPTION OF THE FIGURES

FIG. 1, Table 1, shows a number of polymer blends according to the present invention which produced films exhibiting favorable characteristics, including water resistance.

FIG. 2 shows the results of differential scanning calorimeter (DSC) analysis and the impact on glass transition temperatures of several films prepared from polymer blends according to the present invention.

FIG. 3 shows a silicone elastomer synthetic scheme with modification as described in the present application.

FIGS. 4-6, Tables 2, 3 and 4 show various compositions which are formulated from the silicone elastomer which contains a covalently bonded plasticizer.

### DETAILED DESCRIPTION OF THE INVENTION

The following terms are used to describe the present invention.

In accordance with the present invention there may be employed conventional chemical synthetic methods and techniques within the skill of the art. Such techniques are well-known and are otherwise explained fully in the literature. See, e.g., Smith and March, 2007, “Advanced Organic Chemistry, Reactions, Mechanism and Structure, 6^{th} Edition, Sambrook et al, 2001.

Where a range of values is provided herein, it is understood that each intervening value, to the tenth of the unit of the lower limit unless the context clearly dictates otherwise (such as in the case of a group containing a number of carbon atoms), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are described.

It is to be noted that as used herein and in the appended claims, the singular forms “a,” “and” and “the” include plural references unless the context clearly dictates otherwise.

Furthermore, the following terms shall have the definitions set out below. It is understood that in the event a specific term is not defined in the present specification, that term shall have a meaning within its typical use within context by those of ordinary skill in the art.

The term “compound”, as used herein, unless otherwise indicated, refers to any specific chemical compound such as a monomer, emulsifier, additive, component, etc. disclosed herein. Within its use in context, the term generally refers to a single compound (such as a monomer, emulsifier or similar reactant) which is capable of reacting and forming a covalent bond with a monomeric mixture, an oligomer or polymer as otherwise described herein. In certain instances the term may also refer to stereoisomers and/or optical isomers (including racemic mixtures) or enantiomerically enriched mixtures of disclosed compounds.

The term “composition” refers to a polymeric composition, including compositions according to the present invention such as polymeric compositions and/or personal care compositions and within context, thermoplastic resins, elastomers and/or emulsifiers, which may be used to produce final film-forming compositions according to the present invention.

The term “personal care composition” or “personal care product” is used to describe a chemical composition used for the purpose of cleansing, conditioning, grooming, beautifying, or otherwise enhancing the appearance of the human body. Personal care products include skin care products, cosmetic products, antiperspirants, deodorants, perfume, toiletries, soaps, bath oils, feminine care products, hair-care products, oral hygiene products, depilatories, including shampoos, conditioners, hair straightening products and other hair care products, color cosmetics such as lipstick, creams, make-up, skin creams, lotions (preferably comprised of water-in-oil or oil-in-water emulsions), shave creams and gels, after-shave lotions and shave-conditioning compositions and sunscreen products, among numerous others.

In certain personal care compositions, molded strips can be formulated. These strips can be adhered to a plastic or metal substrate. This molded strip once adhered to the plastic or metal can be referred to as an “Applicator” Delivery of the product onto a surface is a function of passing the metal or plastic which has a molded or gelled strip composed of a crosslinked thermoset and a water based thermoplastic over the surface of the desired application area. Moisture from the surface will activate or release the components of the molded strip onto that surface. A shaving razor would be an example of this mode of delivery. Plastic or metal can also be coated with the thermoplastic+ thermoset to reduce friction. A preferred embodiment would be a comb or brush that has a film adhered to the teeth of the comb or the bristles on a brush. Reduction of friction can also be complimented by the currents inventions capacity to exude/deliver esters, oils, hydrophilic actives or hydrophobic actives. Reduction of friction can be used to optimize delivery of a formulated product onto a substrate such as skin, hair, nail, leather, wood, plastic, metal, etc. This embodiment can be used in brushes that are used to deliver a formula onto the eye lashes. Mascaras are optimized by matching the formulated product to a brush. Coating the bristles of a mascara or eye brow brush may be used to optimize delivery of the product onto it's intended substrate.

Products formulated using the current inventions disclosure can have Newtonian or Non-Newtonian properties. The product form can be Newtonian, flowing like a liquid. Typically low viscosity fluids are considered Newtonian. They respond to gravity or simply put, they will flow or pour from a vessel. Non-Newtonian means the opposite does not flow or respond to gravity therefore they can be described as pastes or gels.

These Newtonian or Non-Newtonian attributes allow for products that are formulated with this inventions disclosures to be delivered or applied to a substrate with the use of various dispensing systems, applicators, or brushes. The products can be delivered by a lip gloss applicator. This applicator can also be used on the skin. Typical lip gloss applicators will be comprised of a plastic rod that has an application surface at the end of this rod. This applicator has an absorbent tip that can be made from many types of material such as cotton or other synthetic compsoitions. This tip could also be a rubber applicator like a spatula.

The product can be applied with brushes such as described previously. The brush can be treated with the current inventions composition or it can be untreated too. The brush can be used on all substrates such as skin, lips, hair, nail, wood, plastic, etc.

The product can be delivered in a tube that has an orifice of varying dimensions at one end. Once the tube is squeezed product can be delivered onto any substrate. Typical delivery of creams and lotions are applied in this manner. There is a surprising aspect of the current invention. The current invention claims a fast set time or dry time. Due to this claim we can use a tube to extrude a bead or tube of product that can dry and form into a solid extrudable application of the product. This can be used to fill fine lines or to accentuate eye lashes.

The product can also be extruded from a double barrel syringe that has a mixing tip at the end of the syringe. One barrel would hold the water based thermoplastic and the adjacent barrel would hold the cross linked silicone thermoset. Pressing down on the syringes plunger will dose an amount of each ingredient. This will deliver the two ingredients into the mixing tip of the syringe and an emulsion can be formed in this manner. This could be helpful if an active would be unstable in an emulsion made using conventional techniques. The mixing tips at the end of the syringe can have a series of baffles that could aid in the mixing of the two phases, Thermoplastic and thermoset phases.

The product can be placed in a jar and application could be fingers, metal, plastic, or a wood applicator.

Products made with the current invention can be made to increase cohesiveness in the formula. Increasing the cohesive aspect of the formula can result in a product that resembles a semi-rubber mass. This would be a Non-Newtonian type product that would apply by wiping an applicator to the surface of the product mass. Applicator can be fingers, plastic, wood, metral, or elastomeric. This mass would yield an amount of product. This amount of product is determined by the cohesiveness of the formula. Cohesiveness can be directly related to the level of Non-Newtinian behavior present in the product mass.

The current invention contains water and can sometimes contain volatile solvents. This allows formulators the ability to produce a very cohesive or Non-Newtonian product that can be poured or extruded into a container, vessel, pan, etc. This vessel containing the product can then be heated or baked. Heating this product in a vessel will volatilize all solvents and increase cohesiveness to the point where the system is now a non-volatile as well as non-aqueous. This results in the product state conversion from an emulsion cream or paste to a solid. This solid can contain all ingredients described in the present invention.

The products or emulsions made with the current invention can also be applied onto woven or non-woven fibers. Wipes are typically made from non-woven fibers and can contain most ingredients used in the personal care or house hold industry. This could lead to delivery of the product onto skin, hair, nails, wood, leather, plastic, metal, synthetic polymers, etc. One aspect is delivery and another would be to reduce friction that could lead to dragginess or irritation associated with the frequent use of wipes.

Woven fibers are typically used in the production of fabrics used in the clothing industry such as shoes or shirts. Applying a product made from the current inventions disclosure can produce fabrics that are water resistant, stain resistant, and may even change the texture or feel of the fabric. One surprising aspect of the current invention is the ability to forma a film with a very quick set time (10-30 seconds) while containing a very high percentage of a non volatile solvent such as hydrocarbons, esters and or silicone oils.

Woven and non-woven fabrics can be used in bandages or gauze. The current invention discloses the ability for products to deliver actives as well as a high level of water resistance and chemical resistance. These are important attributes for the dressing of a wound. The films made from this current invention are non-occlusive so are ideal for dressing wounds.

Personal care products according to the present invention comprise an admixture or polymer of a thermoplastic resin and an elastomer, preferably a thermoset silicone elastomer as otherwise described herein and an additional component selected from the group consisting of water, an aqueous solvent (e.g. alcohol or other compatible solvent), a non-aqueous solvent, emollients, humectants, oils (polar and non-polar) conditioning agents, surfactants/emulsifiers, thickeners/thickening agents, stiffening agents, emulsifiers, medicaments, fragrances, preservatives, deodorant components, anti-perspirant compounds, skin protecting agents, pigments, dyes, coloring agents, sunscreens, waxes, sunscreens, AP-DEO ingredients, clays and minerals, etc. and mixtures thereof, among others.

In certain preferred embodiments, personal care products according to the present invention comprise about 0.01% to about 90%, about 0.05% to about 80%, about 0.1% to about 75%, about 0.5% to about 50%, about 1% to about 35%, about 0.5% to about 25% by weight of an admixture of a silicone elastomer and a thermoplastic resin, in certain aspects a silicone elastomer and a polyurethane or a crosslinked silicone elastomer/polyurethane polymer as the silicone elastomer in combination with a thermoplastic resin, the remainder of the composition comprising at least one additional component selected from the group consisting of water, an aqueous solvent (e.g. alcohol or other water compatible solvent), a non-aqueous solvent, emollients, humectants, oils (polar and non-polar), conditioning agents, emulsifiers, surfactants, thickeners, stiffening agents, medicaments, fragrances, preservatives, deodorant components, anti-perspirant compounds, skin protecting agents, pigments, sunscreens and mixtures thereof, among others.

The term “subject” or “patient” is used throughout the specification within context to describe an animal, generally a mammal and preferably a human, on whose surfaces personal care compositions according to the present invention are deposited to provide films according to the present invention.

The term “surface” or “substrate” is used to describe the surface upon which compositions according to the present invention are deposited in order to provide films which exhibit the characteristics of water resistance, wear resistance and transfer resistance. Typical substrates can be skin, hair, nails, fabric, leather, wood, glass, metal, rubber, etc. “Keratinous tissues” refer to surfaces of the skin, hair and/or nails of a subject or patient.

The term “effective” is used herein, unless otherwise indicated, to describe an amount of a compound or composition which, in context, is used to produce or effect an intended result, whether that result relates to the formation of a film on a surface or the inclusion of a component into a composition or formulation to effect a particular result. The term effective subsumes all other effective amount or effective concentration terms which are otherwise described or used in the present application.

The term “thermoplastic resin” or “water dispersible thermoplastic resin” is used to describe one of the components of compositions according to the present invention. Exemplary thermoplastic resins for use in the present invention include the following: acrylonitrile butadiene styrene polymers (ABS), polyacrylic or poly(meth)acrylic resins (PMA), celluloid cellulose acetate, cyclic olefin copolymers (COC), ethylene-vinyl acetate (EVA), ethylene vinyl alcohol (EVOH), fluoroplastics (e.g. PTFE, FEP, PFA, CTFE, ECTFE, ETFE), acrylic/PVC copolymer, liquid crystal polymer (LCP), polyacrylonitrile (PAN or acrylonitrile), polyoxymethylene (POM), polyamide (nylon), polycarbonate, polyamide-imide (PAI), polyaryletherketone (PAEK), polybutadiene (PBD), polybutadiene/styrene copolymers, polybutadiene/acrylic copolymers, polybutadiene/acrylamide copolymers, polybutylene (PB), polybutylene terephthalate (PBT), polycaprolactone (PCL), polychlorotrifluoroethylene (PCTFE), polyethylene terephthalate (PET), polyhydroxyalkanoates (PHAs), polyketone (PK), polyester, polyethylene (PE, both low and high density), polyetheretherketone (PEEK), polyetherketoneketone (PEKK), polyaryletherketone (PAEK), polyetherimide (PEI), polyethersulfone (PES), chlorinated polyethylene (CPE), polyimide (PI), polylactic acid (PLA), polymethylpentene (PMP), polyphenylene, polyphenylene oxide (PPO), polyphenylene sulfide (PPS), polyphthalamide (PPA), polypropylene (PP), polystryrene (PS), polysulfone (PSU), polytrimethylene terephthalate (PTT), polyurethane (PU), polyvinyl acetate (PVA), polyvinyl chloride (PVC), polyvinylidene chloride (PVDC) and styrene-acrylonitrile, among others.

Exemplary thermoplastic resins for use in the present invention include the following:
BASF (Trademark Name) INCI Name
Luviflex Silk PEG/PPG-25 Dimethicone/Acrylates Copolymer
Luviflex Soft Acrylates Copolymer
Luviset Clear VP/Methyl Acrylamide/Vinyl Imidazole Copolymer
Luviset PUR Polyurethane-1
Luviset Shape Polyacrylate-22
Luviset CAN VA/Crotonates/Vinyl Neodecanoate Copolymer
UltraHold Strong Acrylates/t-Butylacrylamide Copolymer
Ultrahold 8 Acrylates/t-Butylacrylamide Copolymer
Bayer
Baycusan C1000 Polyurethane-34
Baycusan C1003 Polyurethane-32
Baycusan C1004 Polyurethane-35
Baycusan C1008 Polyurethane-48
Dow
Acudyne DHR Acrylates/Hydroxyesters Acrylates Copolymer
Acudyne 180 Acrylates/Hydroxyesters Acrylates Copolymer
Acudyne 1000 Acrylates/Hydroxyesters Acrylates Copolymer
Acudyne LT-120 Acrylates/C-12 Succinates/Hydroxy Acrylates Copolymer
Syntran PC 5100 Polyacrylate 21 (and) Acrylates/Dimethylaminoethyl Methacrylate
Copolymer
Syntran PC 5117 Polyacrylate-18 (and) Polyacrylate-19
Syntran PC 5205 Polyacrylate-15 (and) Polyacrylate-17
Syntran PC 5208 Polyacrylate-15
Syntran PC 5400 Ammonium Acrylates Copolymer
Syntran PC 5227 Polyacrylate-15 (and) Polyacrylate-17
Syntran 5760 Styrene/Acrylates/Ammonium Methacrylate Copolymer
LCW Sensient
Covacryl A15WP Acrylates Copolymer, Phenoxyethanol
Covacryl P12 Acrylates Copolymer
Covacryl MS 11 Acrylates Copolymer, Phenoxyethanol, Methyl Paraben, Ethyl Paraben, Butyl Paraben, Propyl Paraben, Isobutyl Paraben
Covacryl 14 WP Acrylates Copolymer, Phenoxyethanol
Covacryl MT 10 Acrylates/Ethylhexyl Acrylate Copolymer

Molecular weight of thermoplastic resins for use in the present invention will vary considerably depending upon the elastomer chosen, its characteristics including its crosslinking, but generally ranges from about 10,000 to greater than 1,000,000 (up to about 10,000,000 or more), often about 25,000 to about 1,000,000, about 50,000 to about 1,000,000, about 100,000 to about 750,000, about 150,000 to about 500,000.

In embodiments according to the present invention, water borne thermoplastic polymers are typically dispersed at 10%-60% polymer by weight in water. Water borne polyurethane thermoplastic polymers are typically dispersed at 30% polymer by weight in water.

The terms “elastomer” “silicone elastomer and crosslinked silicone elastomer” and “thermoset” are used to describe a component in the polymeric compositions according to the present invention. An elastomer is a polymer, preferably and often non-vulcanised which exhibits viscoelasticity (“elasticity”), generally having low Young's modulus and high yield strain compared with other materials. The term, which is derived from elastic polymer, is often used interchangeably with the term rubber, although rubber is the preferred term when referring to vulcanisates, distinguishable from silicone elastomers generally used in the present invention. In elastomers, each of the monomers which link to form the polymer is usually made of carbon, hydrogen, oxygen and/or silicone. Silicone elastomers for use in the present invention are preferred. Elastomers are amorphous polymers existing above their glass transition temperature, so that considerable segmental motion is possible. At ambient temperatures (ie., about room temperature, elastomers are relatively soft and deformable). Their primary uses are for seals, adhesives and molded flexible parts. Molecular weight of elastomers will vary considerably depending upon the elastomer chosen, its characteristics including its crosslinking, but generally ranges from about 10,000 to greater than 1,000,000 (up to about 10,000,000 or more), often about 25,000 to about 1,000,000, about 50,000 to about 1,000,000, about 100,000 to about 750,000, about 150,000 to about 500,000.

Cross linked silicone thermoset elastomer dispersion can contain about 1%-70% elastomer dispersed in solvent. It is noted that cross linked silicone thermoset elastomers if internally plasticized could be used as a liquid polymer at 100% (i.e., “neat”) without solvent dilution as the elastomer component used in compositions according to the present invention.

There are two general types of elastomers, thermoset and thermoplastic, although a third type, a non-thermoset elastomer (based upon silicone chemistry, for example, polydimethylsiloxane and related polysiloxanes) may also be included as an elastomer. Thermoset elastomer resins (generally, based upon silicone chemistry in the present invention) require curing (heat, chemical reaction or irradiation) and the curing process provides for crosslinking to produce covalent bonds. Once a thermoset elastomer is cured (crosslinked), the polymer cannot be melted. Examples of thermoset resins include natural rubbers, silicone cross polymers, styrene-butadiene copolymers, among others. Thermoplastic elastomer resins (referred to under the general heading thermoplastic polymers or thermoplastic resins to distinguish this component from the silicone elastomer component also used-thus a thermoplastic elastomer as used herein is a subset of a thermoplastic resin as described herein) require no curing or crosslinking. The bonds are generally formed by hydrogen bonding and/or dipole-dipole interactions and the polymers can be melted to a liquid state and cooled to a solid state quite readily. Examples of thermoplastic elastomer resins include thermoplastic urethanes and thermoplastic olefins.

There are six (6) main thermoplastic resins. These are:
styrenic block copolymers, exemplified by styrene/butadiene rubber (SBR);
polyolefin blends (TPOs), exemplified by polyethylene and polypropylene waxes;
elastomeric alloys, exemplified by melt processable rubbers;
thermoplastic polyurethanes (TPUs), exemplified by Polyderm PPG-20;
thermoplastic copolyesters, exemplified by polyethylene terephthalate;
thermoplastic polyamides, exemplified by nylon.

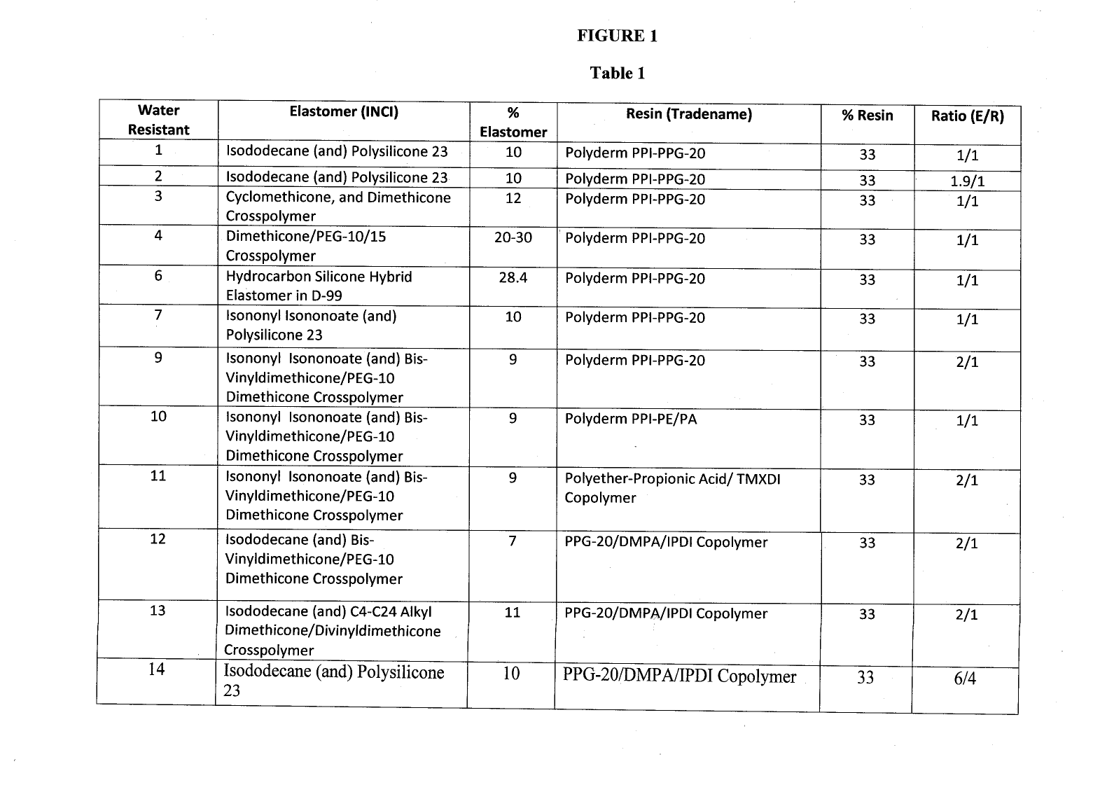

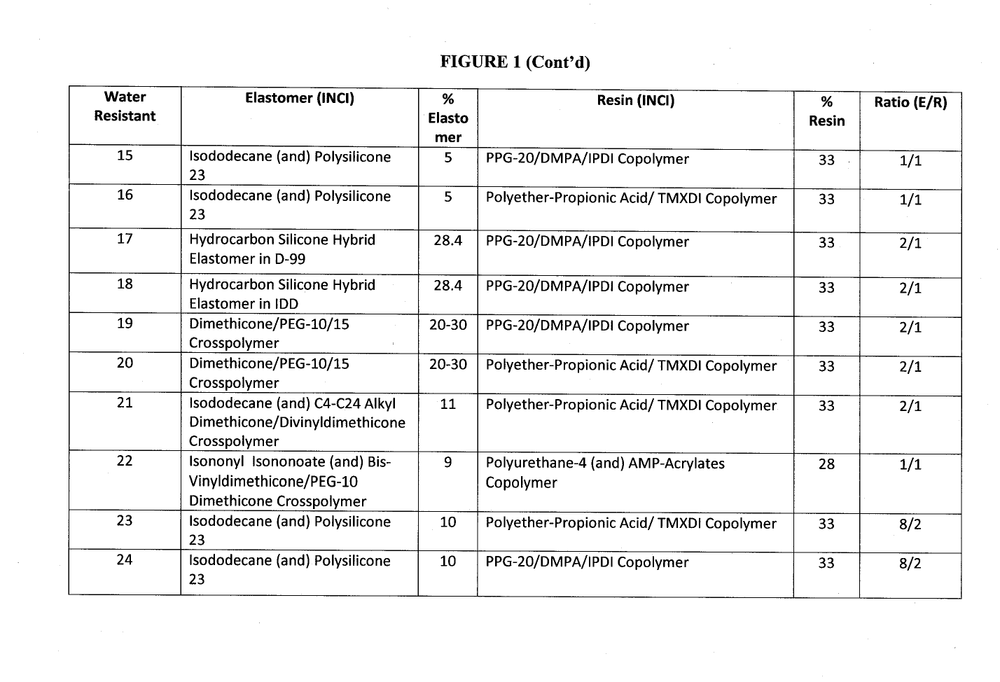

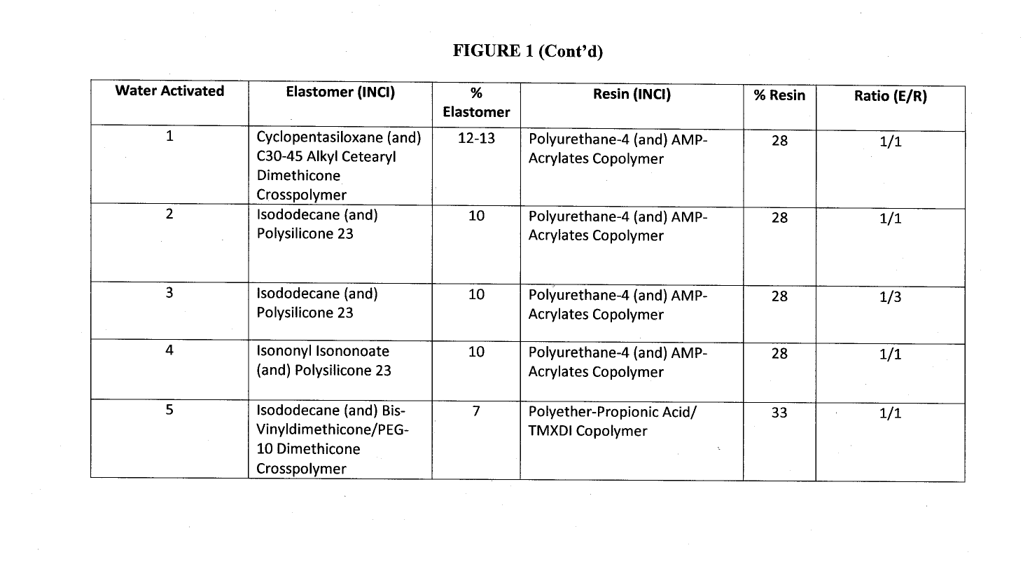

Thermoplastic polymers for use in the present invention are preferably water based thermoplastic elastomers, which are generally dispersible in an aqueous solvent (e.g. water/ethanol, water/isopropanol or water in another solvent). They contain hydrophilic functional groups including ethoxylates, propoxylates, amines, acids and their corresponding salts, or they may be self-emulsifying as described herein. Functionality of these resins dictates the level of hydrophilicity of the polymer which ultimately influences the ability of the thermoplastic film form to emulsify thermoset elastomers. Reduction of surface tension allows for a better integration of a thermoplastic elastomer into the thermoset elastomer for a better integration, which leads to homogenous film formation. Exemplary thermoplastic elastomers which may be used in the present invention including, for example, Polyderm PPI-PE/PA, Polyderm PPG-20, among numerous others, including those which are used in the blends which are presented in Table 1, FIG. 1, attached hereto.

In certain embodiments according to the present invention, the inclusion of propanolamines as volatile emulsifiers (generally, within the thermoplastic dispersion) are included in compositions according to the present invention. Polyderm PPI-PE/PA, INCI: Polyurethane 18 contain volatile propanolamines. Propanolamines are used to solubilize waterborne polymers such as Polyurethane 18. Ther are non-volatile and volatile types. Polyurethane 18 is solubilized with a volatile propanolamine this creates a fast setting waterborne resin that is extremely water resistant. The volatility of the this solubilizers ensures that the cast film does not contain residual solubilizer/emulsifier and the film is now made water resistant. Water borne resins containing non-volatile solubilizers/emulsifiers when dry create a films containing these solubilizers/emulsifiers, thus rendering the film less water resistant. An example of volatile propanolamines are 2-dimethylamino-2-Methyl-1-Propanol sold by DOW under the tradename DMAMP-80, among others. Ionic salt formation of a thermoplastic polymer may be used to diperse the thermoplastic in water.

Preferred elastomers for use in the present invention include silicone thermoset elastomers. The term “silicone thermoset elastomer” describes a polyorganosiloxane polymer, which is often and preferably crosslinked. Elastomers to be included in the present invention are non-vulcanized. By non-vulcanized, the inventors mean that the elastomer is not subjected to a vulcanization step at any time during the process of preparing the elastomer. Vulcanization or vulcanisation is a chemical process for converting rubber or related polymers into more durable materials via the addition of sulfur or other equivalent “curatives” or “accelerators”, such as, for example sulfur systems, peroxides and metallic oxides to make these polymers particularly hard and long-lasting. These additives modify the polymer by forming crosslinks (often sulfur bridges) between individual polymer chains. Vulcanized materials are less sticky and have superior mechanical properties and form the basis for hard rubbers (tires) and industrial materials. The elastomers useful in compositions according to the present invention avoid vulcanization procedures.

Preferred silicone thermoset elastomers for use in the present invention include those described in U.S. Pat. No. 6,939,686, the entire contents of which is incorporated by reference herein. The term “hydrophilic silicone thermoset elastomer” describes a polyorganosilixoane polymer which is crosslinked and contains appreciable quantities of hydroxyl groups or other moieties which instill hydrophilicity to produce a material that is primarily hydrophobic in character, but is sufficiently hydrophilic in order to be compatible with water and other polar solvents. In certain aspects, especially where the silicone elastomer resembles rubber (resumes its original shape when a deforming force is removed), the material is crosslinked with or incorporates quantities of polybutadiene or a multi-unsaturated polyurethane. The term “hydrocarbon silicone crosslinked elastomer” or “hydrocarbon silicone crosslinked polymer” describes a multi functional compound which may or may not be a polymer (for example, polybutadiene or a multi-unsaturated polyurethane, more preferably polybutadiene) which is crosslinked (or chain-extended) with a bis-hydrosilane terminated polysiloxane and exhibits favorable characteristics of gelation, solubility and stability for use in the present invention. These are generally described in international PCT application PCT/US2012/0843, filed Mar. 29, 2012, which is incorporated by reference herein.

Some preferred thermoset elastomers for use in the present invention include the following (all available from Alzo International, Inc., Sayreville, N.J.):
NuLastic™ Silk E D-99 LSA (Proposed) Isononyl Isononanoate (and) Polysilicone 23
NuLastic™ Silk E DM LSA (Proposed) Dimethicone (and) Polysilicone 23
NuLastic™ Surfa D99 Isononyl Isononanoate (and) Bis-Vinyldimethicone/PEG-10 Dimethicone Copolymer
NuLastic™ Silk MA, DM Dimethicone (and) C4-24 Alkyl Dimethicone/Divinyldimethicone Crosspolymer.

The polyorganosiloxane polymer (bis-hydrosilane silicone polymer) which is crosslinking (or chain-extending) the hydrocarbon according to the present invention may vary significantly in chemical composition but often is a polymeric composition comprised of repeating polysiloxane units

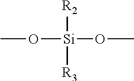

units, where R² and R³ are each independently a C₁-C₁₀ alkyl (preferably C₁-C₃ alkyl, more preferably methyl) (as described below), and optionally, in a small number of instances in certain embodiments as otherwise described herein, Si—H groups or hydroxyl groups, and may vary in average molecular weight M_{w} from about 1,000 to about 1,500,000 or more, preferably about 1,000 to about 100,000, more preferably about 2,500 to about 25,000 or more, depending upon the final viscosity and other characteristics desired.

Silicone thermoset elastomers may also comprise allyl alcohol ethoxylate units, polyurethane units and/or hydrocarbon units as otherwise described herein. In certain embodiments, silicone crosslinking agents (bis-hydrosilane terminated polyalkylsiloxanes) described herein may comprise as little as 0.25% and as much as 98% by weight of the final silicone crosslinked elastomer, especially in polymers comprising a multifunctional hydrocarbon compound, but in preferred aspects the silicone crosslinking agent comprises about 0.5% to about 90%, often 1% to about 25% of the final silicone crosslinked elastomer, about 0.1% to about 25%, about 0.25% to about 20%, about 0.5% to about 15%, about 1% to about 10% by weight of the final silicone crosslinked elastomer. In certain other aspects, a polyurethane polymer may be added to (admixed), rather than polymerized with, the silicone elastomer.

Silicone polymers according to the present invention which are used to produce silicone crosslinked elastomers preferably comprise Si—H group(s) at each of the distil ends of the elastomer (e.g. bis-hydrosilane polydimethylsiloxane) which are capable of crosslinking with multi vinyl functional crosslinking agents, including multi vinyl functional hydrocarbons as otherwise described herein (e.g. polybutadiene, unsaturated polyurethane, among others) or alternatively, polysiloxane polymers.

In certain embodiments, an allyl alcohol ethoxylate (or an alkylene ethoxylate) may optionally comprise (in the final silicone elastomer polymer) an amount of about 0.01% to about 7.5%, about 0.05% to about 5%, about 0.1% to about 1% by weight of the monomers/polymers which ultimately form certain embodiments of the silicone crosslinked hydrocarbon elastomer according to the present invention. The inclusion of allyl alcohol ethoxylate may increase the hydrophilicity of the final silicone crosslinked hydrocarbon elastomers according to the present invention. In certain other aspects, a polyurethane polymer also may be added to the silicone elastomer alone, in combination with a allyl alcohol ethoxylate and/or to the multifunctional unsaturated hydrocarbon crosslinkable agent and reacted with the bis-hydrosilane polyorganosiloxane polymer to provide final hydrophilic silicone crosslinked elastomers. The polyurethane polymer comprising (when optionally present) about 0.01% to about 15%, about 0.05% to about 10%, about 0.05% to about 5% or more by weight of the final polymeric composition in order to provide a further hydrophilic/skin adhering component, solubilizer or UV absorbing component.

Accordingly, the final silicone crosslinked elastomeric polymers may optionally include multifunctional hydrocarbon, allyl alcohol ethoxylate and/or polyurethane units to increase flexibility (hydrocarbons), hydrophilicity (allyl alcohol ethoxylate) or, in the case of polyurethanes, hydrophilic, skin-adherent, solubilizing or UV absorbing qualities of the final polymers, which according to the present invention may be crosslinked with a multifunctional hydrocarbon (polybutadiene) or a bis-hydrosilane terminated Polysiloxane compound or pendant hydrosilane Polysiloxane (e.g., the reaction preferably occurring between the olefinic groups on the multiple unsaturated hydrocarbon and the Si—H groups and, in some cases, optional alkenyl groups on the crosslinking silicone polymer). In certain embodiments, vinyl terminated polydimethylsiloxane can be replaced by a vinyl terminated hydrocarbon such as 1, 7 octadiene and/or 1, 5 hexadiene. Using a smaller molecular weight bis-vinyl will yield a glassy (hard) gel instead of a soft (rubbery) gel. Harder silicone rubbers can be micronized into a solid state since the gel can be made without solvnets. Optionally, the bis-hydrosilane polydimethylsiloxane or pendant hydrosiliane Polysiloxane may be reacted with an unsaturated polymeric silicone compound, an alpha olefin and/or an allyl alcohol ethoxylate prior to crosslinking with the multiply unsaturated hydrocarbon compound. For example, polydimethylsiloxanes with several pendant hydrosilane groups may be used to introduce an allyl alcohol ethoxylate (each allyl alcohol monomer preferably containing from 5 to about 100, about 10 to about 50, about 15 to about 45, about 10 to about 65, about 15 to about 25, about 50 to about 100, about 65 to about 85, about 75 ethoxylate/ethylene glycol units) monomer into the final silicone crosslinked hydrocarbon polymer. These groups can also be used to introduce polyurethane or polyester compounds having the appropriate unsaturated group. Alternatively, the hydrophilic silicone elastomer (hydrophilic through introduction of allyl alcohol ethoxylate groups) and/or polyurethane or polyester may simply be admixed without further crosslinking/polymerization.

In certain embodiments of the present invention, the final silicone thermoset elastomer is prepared from a reaction mixture which comprises a hydrosilane terminated polydimethylsiloxane polymer as described above (which may optionally further comprise an allyl alcohol ethoxylate group as described herein and/or a reactive polyurethane or polyester wherein the hydrosilane terminated polydimethylsiloxane and the allyl alcohol ethoxylate and/or polyurethane or polyester are covalently linked) as a crosslinking agent. This crosslinking agent may then be reacted with a crosslinkable polymer silicone compound or a multifunctional unsaturated hydrocarbon such as polybutadiene as described herein. The polybutadiene itself may be optionally mixed or combined with an allyl alcohol ethoxylate and/or a polyurethane or polyester prior to reaction with the hydrosilane terminated polydimethylsiloxane crosslinking agent to form the final silicone crosslinked hydrocarbon polymer according to the present invention. Thus, silicone elastomers according to the invention may comprise the reaction product of a crosslinking silicone polymer as otherwise described hereinabove that contains hydrosilane groups at the distil ends of the polysiloxane, as well as an optional allyl alcohol ethoxylate component and/or an optional polyurethane or polyester component. Each of the optional allyl alcohol ethoxylate component and the optional polyurethane or polyester component independently comprise about 0.1% to about 75%, about 0.5% to about 50%, about 1% to about 10% by weight of the bis-hydrosilane polydimethylsiloxane crosslinking agent which may be used without further modification or reacted with the multifunctional hydrocarbon polymer to provide additional modified silicone thermoset elastomers for use in the present invention.

Alternatively, the final silicone thermoset elastomers may comprise the reaction product of a crosslinking silicone polymer as otherwise described herein (i.e., without allyl alcohol ethoxylate and/or a polyurethane) with a multiple unsaturated hydrocarbon (e.g., polybutadiene) which may optionally include an allyl alcohol ethoxylate and/or a polyurethane as described above (preferably comprising about 0.01% to about 7.5%, about 0.05% to about 5%, about 0.1% to about 1% by weight of the multifunctional hydrocarbon).

For preparation of a silicone thermoset elastomer which contain a bonded polyurethane to optionally instill at least a portion of hydrophilic, self-adhering, solubilizing and/or UV absorbing character to the final silicone elastomer, the polyurethane compound comprises about 0.01% to about 7.5%, about 0.01% to about 5%, about 0.05% to about 1% of the final silicone thermoset elastomer.

In still other elastomeric embodiments, the bis-hydrosilane polydimethylsiloxanes (silicone polymer crosslinkers) which are used to prepare silicone thermoset elastomers according to the present invention have the following structure:

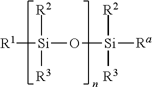

Where R¹ and R^{a} are each independently H groups;

Each R² and R³ is independently a C₁-C₁₀ alkyl group (preferably C₁-C₃ alkyl, preferably methyl); and

n is from 5 to 50,000, about 10 to about 25,000, about 100 to about 10,000, about 100 to about 5,000, about 200 to about 5,000, about 500 to about 2500.

In still other elastomeric embodiments, the polyorganosiloxane polymers (silicone elastomer compounds) which may be used to prepare hydrophilic silicone elastomers (in combination with allyl alcohol ethoxylate and/or polyurethane and/or multifunctional hydrocarabon compounds), according to the present invention have the following structure:

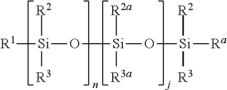

Where R¹ and R^{a} are independently H, an optionally substituted C₁-C₆ alkyl group (substitution with OH or a C₁-C₃ alkyl group which itself may be optionally substituted with a hydroxyl group) or an optionally substituted C₂-C₆ alkenyl group (which term may include an acrylate or methacrylate group);

Each R² and R³ is independently H, OH, or a C₁-C₃ alkyl group (preferably a C₁-C₃ alkyl group, preferably a methyl group), preferably R² and R³ are both C₁-C₃ alkyl groups, preferably both are the same C₁-C₃ alkyl group, preferably methyl groups;

Each R^{2a} and R^{3a} is independently H, OH, or a C₁-C₃ alkyl group, preferably at least one of R^{2a} or R^{3a} is H and the other is a C₁-C₃ alkyl group;

n is from 5 to 50,000, about 10 to about 25,000, about 100 to about 10,000, about 100 to 5,000; about 500 to 5,000; about 500 to about 2,500; about 100 to about 1,000, about 150 to about 1,000; and

j is from 0 to 50, preferably 1 to about 25, about 1 to 4, about 2 to 15 about 3 to 10; about 5 to 10.

In still other embodiments of the present invention, silicone thermoset elastomers may be formed by reacting a polysiloxane polymer which contains two Si—H bonds at distil ends of the molecule (a bis hydrosilane polydialkylsiloxane as otherwise described herein) with a crosslinking agent or other agent, including multifunctional hydrocarbon (e.g. polybutadiene), each of which is reactive with a Si—H group. The multifunctional hydrocarbon may vary in size, but generally ranges in size from a molecular weight of several hundred to 25,000 or more, with a preferred molecular weight range of at least about 500 to about 10,000, about 1500-7500, about 2,000-5,000 or often about 2500.

In certain preferred aspects, the polysiloxane polymer described above is according to the chemical structure:

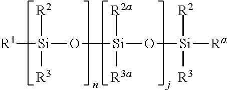

Where R¹ and R^{a} are independently H, an optionally substituted C₁-C₆ alkyl group or an optionally substituted C₂-C₆ alkenyl group;

Each R² and R³ is independently H, OH, or a C₁-C₃ alkyl group;

Each R^{2a} and R^{3a} is independently H, OH, or a C₁-C₃ alkyl group,

n is from 5 to 50,000, and

j is from 0 to 50, wherein the polysiloxane polymer contains about 4 to about 25 Si—H groups. These Si—H groups may be used to react with vinyl groups (carbon-carbon double bonds) in a number of component compounds (e.g., plasticizers, hydrocarbons, polyurethanes, allyl alcohol ethoxylates, etc.) in order to covalently bind the component to the silicone backbone of the polysiloxane polymer.

The following is a list of possible combinations that can be used to create silicone cross linked elastomers:

1. Bis-vinyl silicones+ silanic hydrogen (silicone pre-polymer containing Si—H groups where each reactant is available at different molecular weights);
2. Combination of #1, above mixed molecular weights, ex. a bis-vinyls at different molecular weights+ silanic hydrogen where a >1;
3. Combination #1 mixed molecular weights, ex. Bis-vinyl+ b silanic hydrogen at different molecular weights where >1;
4. Combination #1 mixed molecular weights, ex. a Bis-vinyl at different molecular weights+ b silanic hydrogen at different molecular weights. Where the ratios of 2 and b are a>1 and b>1;
5. Bis-vinyl silicones+ silanic hydrogen+ a olefins (each reactant is available at different molecular weights);
6. Combination #5 mixed molecular weights, same as in combinations 2-4, above, including mixed molecular weights. Ratios of the a olefins can be >1;
7. Bis-vinyl silicones+ silanic hydrogen+ olefins (each reactant is available at different molecular weights and isomers of olefins);
8. Combination #7, same as in combinations 2-4 including mixed molecular weights and isomers of olefins. Ratios of the olefins can be >1;
9. Bis-vinyl silicones+ silanic hydrogen+ esters containing alkenes (each reactant is available at different molecular weights and isomers of esters containing alkenes);
10. Combination #9, same as in combinations 2-4 including mixed molecular weights and isomers of esters containing alkenes. Ratios of the esters containing alkenes can be >1;
11. Bis-vinyl silicones+ silanic hydrogen+ polyenes (each reactant is available at different molecular weights and isomers of polyenes);
12. Combination #11, same as in Combinations 2-4 including mixed molecular weights and isomers of the polyenes. Ratios of the polyenes containing alkenes can be >1;
13. Bis-vinyl silicones+ silanic hydrogen+ Vinyl silicones (each reactant is available at different molecular weights);
14. Combination #13 mixed molecular weights, same as in Combinations 2-4 including mixed molecular weights. Ratios of the vinyl silicones can be >1;
15. Bis-vinyl silicones+ silanic hydrogen+ polyvinyl silicones (each reactant is available at different molecular weights);
16. Combination #15 mixed molecular weights, same as in Combinations 2-4 including mixed molecular weights. Ratios of the polyvinyl silicones can be >1;
17. Bis-vinyl silicones+ silanic hydrogen+ allyl alcohols (each reactant is available at different molecular weights);
18. Combination #17 mixed molecular weights, same as in Combinations 2-4 including mixed molecular weights. Ratios of the polyvinyl allyl alcohols can be >1;
19. Bis-vinyl silicones+ silanic hydrogen+ vinyl alcohols (each reactant is available at different molecular weights);
20. Combination #19 mixed molecular weights, same as in Combinations 2-4 including mixed molecular weights. Ratios of the vinyl alcohols can be >1.

In certain embodiments, multifunctional silicone elastomers are used, for example, multifunctional silicone elastomers such as the reaction product of Isononyl Isononanoate and Polysilicone 23 {NuLastic Silk-E D99-LSA, available from Alzo International, Inc.) which provides hydrophilicity, organic/silicone compatibility and cationic characteristics. Other preferred multifunctional silicone elastomers such as Shin Etsu's KSG 210 may be used which are silicone elastomers that are polyether modified self emulsifying silicone elastomers. This silicone elastomer contains functionalities with hydrocarbon and silicone compatibility and the elastomer has self-emulsifying qualities as discussed in greater detail herein.

Pursuant to certain of the above-described embodiments, the above-referenced silicone elastomers are reacted with a plasticizer compound which contains a functional group which is capable of reacting with the silicone elastomer to provide internal, chemically bonded (grafted) plasticizer onto the silicone elastomer. In preferred aspects, the silicone elastomer contains a number of Si—H groups at the terminal ends of the polymer and in the backbone of the polysiloxane polymer (generally, at least about 3 Si—H groups up to about 20-25 Si—H groups depending upon the level of plasticizer desired) preferably about 4 Si—H groups to about 10 Si—H groups, about 4 Si—H groups to about 8 Si—H groups, about six Si—H groups) two at the distil ends of the polymer and at least one Si—H group in the backbone of the polysiloxane polymer) such that the polymer may be crosslinked with other polymers to provide crosslinked polymer gels and also have plasticizer grafted onto the reactive Si—H groups (with double bonds of the plastizer component forming a bond with the Si of the reactive Si—H group and carbon of the double bond of the plasticizer). It is noted that as an alternative to Si—H groups reactive with ethylenic double bonds, elastomers can be modified to contain Si-halogen (e.g. Si—Cl) groups which are reactive with hydroxyl groups in the plasticizer, or the plasticizer can be modified to contain functional groups which are reactive with Si—OH groups, thus forming Si—O groups to graft the plasticizer onto the silicone elastomer.

As discussed above, in certain preferred aspects of the present invention, wherein the polysiloxane polymer above containing Si—H groups (also referred to as an activated siloxane polymer) is reacted with at least one plasticizer compound which can be grafted onto activated polysiloxane polymers at Si—H groups, esters and other plasticizer compounds that contain or can be reacted to contain double bonds or other functional groups, alcohols that contain or can be reacted to contain double bonds or other functional groups, or mixtures thereof, may be used. This synthetic approach thus provides “internal” plasticizers according to the present invention. In this embodiment of the invention, the plasticizer compound includes, any plasticizer with functional groups consistent with chemically attaching the plasticizer molecule into an elastomer, and can include for example, cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, Di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂-C₁₃ alkyl lactate, C₁₂-C₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂-C₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxstearate or a mixture thereof. The siloxane polymers so produced may be first crosslinked (to produce higher viscosity compostions including gelled compositions) and reacted with plasticizer compound either before crosslinking or after crosslinking to produce thermoset silicone elastomers which have been chemically modified with at least one internal plasticizer (i.e., a plasticizer which is a separate component from the polysiloxane polymer which itself often has plasticizing characteristics). By providing an integral (covalently attached) plasticizer, the final compositions may instill plasticizer characteristics (e.g. lowering the transition temperature and providing a softer feel on a surface) to the final film while at the same time providing a stronger, longer lasting and more durable film compared to those films which contain plasticizers which are admixed, rather than covalently linked to the elastomer and/or the thermoplastic resin.

Representative plasticizer compounds which can be grafted onto silicone elastomer gels include one or more of the following:

(A) Esters that contain double bonds and can be grafted on silicone gel (e.g. at Si—H groups);
(B) Esters that contain hydroxyl groups and/or contain or can be reacted to contain double bonds;
(C) Alcohols that optionally contain or can be reacted to contain double bonds.

As described above, in each instance the double bond is reactive with a Si—H group of the silicone elastomer described above such that the plasticizer molecule is grafted onto the silicone elastomer at the reactive Si—H group, forming a Si—C bond with the silicone polymer backbone. The following plasticizers are representative of those which can be grafted or readily modified to be grafted onto silicone polymers as described above. In each instance, the plasticizer is an ester or an alcohol of varying length as exemplified below. In certain aspects the ester or alcohol is a volatile short-chain plasticizer or alternatively, the plasticizer can be an ester or alcohol which preferably contains a long chain C₈-C₂₆ optionally substituted (with OH, etc.) hydrocarbon group which instills plasticizing characteristics to the final film composition and the final film which is deposited as a coating on a surface.

### Examples of A

### Dermol CTR: Cetyl Ricinoleate

DID: Diisopropyl Dimer Dilinoleate
DO: Decyl Oleate
GMO: Glyceryl Monooleate
ISE: Isostearyl Erucate
MAHO: Methyl Acetyl Ricinoleate
OE: Oleyl Erucate
OL: Oleyl Lactate
OLO: Oleyl Oleate
PGR: Propylene Glycol Ricinoleate
Waxenol 801: Arachidyl Propionate
Waxenol 822: Arachidyl Behenate
Bernel Ester DCM: Dicapryl Maleate
Bernel Ester DID: Diisopropyl Dimer Dilinoleate
Marrix SF: Di-C12-15 Alkyl Fumarate
Parapel HC: Linoleamidopropyl Ethyldimonium Ethosulphate
Dermol GTR: Glyceryl Triacetyl Ricinoleate
Dermol GRC: Glyceryl Diricinoleate
Polyderm PPI-GRC: Glyceryl Diricinoleate/Copolymer

### Examples of B

### Dermol 20-S Octyldodecyl Hydroxystearate

23-L: C12-C13 Alkyl Lactate
25-L: C12-C15 Alkyl Lactate
CL: Cetyl Lactate
EDG: Ethoxydiglycol
G-7CT: Glycereth-7 Citrate
G-7LC: Glycereth-7 Lactate
ICSA: Isocetyl Salycilate
IDSA: Isodecyl Salycilate
IDO: Isodecyl Oleate
IPM: Isopropyl Myristate
ISL: Isostearyl Lactate
L-45: Glycereth 4.5Lactate
LL: Lauryl Lactate
ML: Myristyl Lactate
NSA: C12-C15 Alkyl Salicylate
PGB: Propylene Glycol Benzoate
PGML: Propylene Glycol Lactate
TDSA: Tridecyl Salicylate
Dermolan GLH: Glycerol-7 Hydroxystearate
Dermowax EGMS: Ethylene Glycol Distearate
Dermowax GMHS: Glyceryl Hydroxystearate
Dermowax GMS: Glyceryl Stearate
Dermowax PGMS: Propylene Glycol Stearate
Bernel Ester TCC: Tricapryl Citrate
Citmol 316: Triisocetyl Citrate
Citmol 320: Trioctyldodecyl Citrate
Hetester ISS: Isostearyl Stearoyl Stearate
Hetester HCA: Glyceryl Triacetyl Hydroxstearate

### Examples of C

### Stearyl Alcohols Reacted with Other Organic Compounds to Produce Products that Contain Double Bonds which can be Grafted onto Silicone Gels

The term “crosslinking” is used to describe the reaction of the silicone polymer with the multifunctional hydrocarbon backbone in the present compositions. It is noted that in instances when the silicone polymer has only two functional groups, e.g. a Si—H group on each of the distil ends of the silicone polymer, the polymer may also be referred to as a chain extender or chain extending agent. However, it will be understood the term crosslinking may be used to refer to the silicone polymer or crosslinker used in the present invention and the reaction of the silicone polymer or crosslinker with the (multiply unsaturated) hydrocarbon.

The term “self-emulsifying” applies to thermoplastic resins and/or elastomers which are used in the present invention. It is noted that the emulsifier is an optional component in compositions according to the present invention and are added in order to emulsify the thermoplastic resin and water (aqueous solvent) and the elastomer and the solvent in which it is solubilized, dispersed and/or gelled. In instances where the external emulsifier is excluded, one or both of the thermoplastic resin and/or the elastomer (including a thermoset elastomer as otherwise described herein) is self-emulsifying.

A self-emulsifying polymer is defined by the presence of an emulsifying moiety that is part of the chemical composition of the thermoplastic resin and in some cases the elastomer (preferably, a thermoset elastomer). The emulsifying moiety of the polymer is what allows the thermoplastic resin to be dispersed in water or aqueous solvent. The emulsifying moiety of the thermoplastic resin is often sufficient to create a thermoplastic resin which can function as an emulsifier (as well as a thermoplastic resin). The blending of a thermoplastic resin and elastomer with sufficient emulsifying moieties can yield a homogenous blend of thermoplastic resin and elastomer which can provide a homogenous blend which can be made into a film exhibiting favorable characteristics without the addition of an external emulsifier.

The self emulsifying property is a function of the ratios of the thermoplasmic resin and the elastomer and the weight ratio of emulsifying moiety in each polymer. Depending upon the amount of emulsifying moiety in each polymer, more of either the thermoplastic resin or the elastomer will increase or decrease the level of emulsifier in the blend. For example, one can choose to include in compositions according to the present invention a silicone elastomer (thermoset) that contains emulsifying properties or not, (NuLastic™ Surfa vs. NuLastic™ MA ID, available from Alzo International, Sayreville, N.J., USA). Blending at least one thermoplastic resin that emulsifies and at least one elastomer (which may be a mixture of a thermoset and/or a thermoplastic elastomer) that is very water soluble may result in enough emulsifier in the blend from the polymers to allow a mixing of the two without the need for an external emulsifier. The selection of the blend components can optimize the emulsion to a level where no external emulsifier is needed, yet the favorable film-forming characteristics will be maintained.

The self-emulsifying moiety in the thermoplastic resin(s) and/or the elastomer(s) used in the present invention ranges in weight from about 0.01% (preferably at least about 0.1%) to about 40%, more often about 0.25% to about 20%, more often about 0.5% to about 15%, about 1% to about 10%, about 1.5% to about 7.5%, about 1% to about 5% by weight of the polymer in which the emulsifying moiety occurs. It is noted that the relative weight percent of emulsifying moiety in the thermoplastic resin and/or elastomer in the composition will reflect a weight percentage of the total weight of thermoplastic resin, elastomer and solvents, and in many instances this later weight percentage will reflect the amount of external emulsifier which may be included in the present invention.

Many of the elastomers, especially silicone elastomers, including silicone thermoset elastomers, do not contain significant hydrophilic content or character which can assist in providing self-emulsifying type character to influence the final film characteristics in combination with the water based thermoplastic resin, but a number clearly do. Determining the hydrophilic content of a given elastomer can assist in identifying the likelihood that the type and amount of an external emulsifier should be added to a composition in order to influence the final form characteristics. Some examples of typical thermoset elastomers and their hydrophilic content are exemplified below.

Hydrophilic content for the following dispersions which roughly equate to the amount of external emulsifier which would be added by virtue of including (note that a figure is given for the hydrophilic content of the dispersion).

| | |
|---|---|
| NuLastic ™ Surfa (9% polymer dispersion) | 0.63% |
| Surfa polymer has a 7% Hydrophilic content | |
| Polyderm PPG-20 (33% polymer) | 3.7% |
| POLYDERM PPG-20 has a 11.1% Hydrophilic content | |
| Polyderm PE/PA (33% polymer) | 3.9% |
| PE/PA has a 11.9% Hydrophilic content | |

In contrast, the following well-known emulsifiers have a hydrophilic content
As follows:

| | |
|---|---|
| Polysorbate 20 | 84% |
| Sorbitan Oleate | 21.5% |

Thermoplastic elastomer (TPE) levels will not work when the percent by weight of a polymer/solvent mixture approaches 25% by weight polymer. Calculating hydrophilic content at 25% results in the following:

POLYDERM PPG-20 (0.25) (3.7)=0.93%

PE/PA (0.25) (3.9)=0.98%

The above levels reflect the inability to create stable emulsions without the use of an external emulsifier.

Similarly, a 30%-50% level of POLYDERM PPG-20 in solvent equates to a hydrophilic content range of 1.11-1.85% and a 30%-50% level of PE/PA equals a hydrophilic content range of 1.17-1.95%. For these two elastomers, a stable self emulsified system is possible at a hydrophilic content of about >1.0% hydrophilic content.

NuLastic™ Surfa (Alzo International) used in experiments contains 7-9% polymer by weight in a solvent.

Thus, for 7% Surfa polymer (0.07) (7)=0.5% hydrophilic content. For 9% Surfa polymer (0.07) (9)=0.63% hydrophilic content.

50%-70% level of NuLastic™ Surfa equals a hydrophilic content of 0.3%-0.4% if one uses a Surfa that contains 9% polymer in solvent.

Thus, contribution from the Surfa is minimal but it adds 0.4% to the values calculated for the TPE above, resulting in the following increases:

POLYDERM PPG-20 will increase from 1.11% to 1.51%, and increase from 1.85% to 2.25%;

PE/PA will increase from 1.17% to 1.57%, and increase from 1.95% to 2.35% Again the slight contribution maintains a >1% hydrophilic content required to achieve self surfactancy.

Water resistance can be influenced greatly by a small change in the hydrophilic content. Typically >2% hydrophilic content is an indication that the films made by such a blend will not be as water resistant as a <2% hydrophilic content, although this general rule is influenced by the chemistry of other components in the polymer.

The term “water resistant” or “water resistance” is used to describe a film which resists water, making it very difficult for the water to penetrate the polymer and modify the shape of the polymer. To test the water resistance of a film, the following procedure is used.

Test procedure. A blend of thermoplastic resin and elastomer is applied to a substrate. It is applied with a spatula to about a 1 mil thickness. The applied film is allowed to dry (about 1-2 hours). The film is removed from the substrate. This film is weighed. The film placed in a dish of water. Water is poured into the dish, at a temperature of 25° C. The water content used is 40 times the weight of the cast film. The film is observed at 10 minutes interval over a 1 hour period. Observations are made visually to determine the water resistance of the film. If, under the test conditions described, the film maintains its shape with no observable swelling or thickening, the film is considered to be extremely water resistant. If the film observably swells/thickens, but does not dissolve or disintegrate, it is absorbing small quantities of water and can be considered moderately water resistant. The film is soft to the touch as well. The present invention includes films which are extremely water resistant or moderately water resistant under the test methodology described. In contrast, if the film dissolves or disintegrates in water it is considered to be water soluble.

The term “uniform” or “homogenous” is used to describe a film which appears consistent (e.g. white) with no clear or translucent voids. Uniformity or homogeneity of a film can be observed through a test procedure wherein a film is cast in the same manner as in the water resistance test, described above. If the film contains no clear or translucent voids, the film is described, for purposes of the present invention, as uniform or homogeneous. The term “homogenous” may also be used to describe the emulsion created by the blend. A blend that does not separate into two layers is considered to be homogeneous. Often, commercial compositions which contain all the components in a single blend (ie., not as a two part mixture to be combined, which is an alternative embodiment of the present invention), are homogenous for a period of at least several weeks, often more than six months or more.

The term “flexible” is used to describe characteristics of films according to the present invention. A film of a thermoplastic resin which excludes elastomer is often a brittle film having characteristics which are inconsistent with the present invention. These films when held at each end and pulled retain their shape. The thermoplastic resin, when combined with an elastomer in the present invention is internally plasticized by grafting the “soft” moieties of the elastomer into the film structure. Thus, compositions according to the present invention yield a cast film that does stretch. Adding more elastomer to the compositions provides a greater stretch to the film, whereas reducing the amount of elastomer reduces the stretch and flexibility of the film.

The term “dries” is used to describe the condition of a film after deposition wherein the volatile solvent has evaporated. Thus, a dry film is a film which contains no volatile solvent after the cast film is allowed to set up on the substrate. The blend of thermoplastic resin and elastomer often contains a volatile solvent, which evaporates or becomes absorbed into the skin. Drying times may vary as a function of the volatility of the volatile solvent, its boiling point and vapor pressure. Often, a film will be dry, after application to a keratinous surface such as the skin within about 5 to about 60 minutes, often less than 30 minutes, preferably about 5-10 minutes. It is noted that pursuant to the present invention a film may be considered dry, even where there is residual water left in the film.

The term “transfer resistance” is used to describe the visual observation of how much of the cast film transfers color (pigments, pearlescent, dyes, etc.) from a first substrate onto a second substrate. In the present invention, the films prepared from the present compositions exhibit transfer resistance compared to prior art films (lipsticks) and in certain preferred embodiments; do not transfer appreciable quantities of color to a second substrate. Transfer resistance is used to describe films that are typically associated with color products, mascaras, lip products, eye shadows, blush, and foundations. Colorless products are generally acknowledged as being transfer resistant. In the case of colorless products, water resistance is the preferred descriptive characteristic. Water resistance implies transfer resistance, but is not identical in physical characteristics. The product is made water resistant so that the actives don't transfer from the applied area. When transfer resistance is referred to, it will be referring to color products (products that contain pigments and/or dyes). It is easier to measure product transfer if the transferrable ingredients are pigmented.

Transfer resistance can be measured in the following manner. This measurement is for products which contain color (pigments and/or dyes) and/or oils and/or actives in the film only. Those films which are neat, i.e., do not contain a pigment/dye, oil and/or active, do not exhibit transfer and the preferred method of characterizing these films is by describing the film's water resistance. A composition according to the present invention is applied and allowed to dry or set up for a time which allows volatile solvent to be evaporated from the film—preferably about 3-5 minutes. A white paper or cloth is pressed on to the area where product has been applied to for a period of at least about a minute. The paper or cloth can be visually inspected for any trace of color that may have transferred from the applied areas. Those films which do not transfer appreciable quantities of color to the paper or cloth as evidenced by the visual inspection of same (no visual transfer) or by change in weight (no appreciable change) of the paper or cloth are said to be transfer resistant. Any appreciable color transferred onto the white surface by visual inspection and/or weight change evidences that the product is not transfer resistant. This method will quantify the transfer of product onto a substrate. Quantifying product transfer in this way is a cumulative measurement of color, oils, or actives that have transferred onto a substrate.

The term “long wear” is used to describe films according to the present invention. Long wearing films are those where the film maintains its integrity on the surface in which it has been applied. Oftentimes, long wearing characteristics are synonymous with transfer resistance for those films which further comprise pigments/dyes, oils and/or active agents. In the present invention, if the film doesn't transfer, it is assumed that it remains where it was applied. Long wearing films, such as lip products generally last about 8-16 hours or longer, including up to 24 hours or longer. No transfer of product over an 8 or 16 hour period indicates that the product is still visible on the applied substrate over that period of time.

The term “Hydroactive Delivery Elastomer” or “Hydroactive Elastomeric Delivery System” or “Hydrolastomeric Delivery” or “Hydroactomeric Delivery” is used to describe the delivery of a bioactive agent from a film pursuant to the present invention onto a keratinous surface for absorption into a patient or subject. Active delivery can be measured in the following manner.

A composition according to the present invention comprising a blend of thermoplastic resin and elastomer is made as otherwise described in the present application. FD&C Blue #1 (as a proxy for the active) may be mixed into the blend. The dye is water soluble and can be measured in the Visible range using a UV-Vis Spectrophotometer.

The blend contains the Blue #1 dye in a weight percentage ranging from less than 1% to about 10% by weight.

A film is cast and allowed to dry (0.5-1 hour)

The cast film is placed in a jar of water.

The water can be analyzed for dye release from the film using a UV-Vis Spectrophotometer.

An aliquot of water is removed at several time intervals and measured over that period of time. The data generated will reflect a timed release of Blue #1 into the water which can be measured using UV analysis. Alternatively, if it was required to measure an active that does not absorb in the UV-VIS range, GC analysis can be utilized, using the same procedure described above. Upon release of active, the water can be analyzed using a GC and consequently, the active release into water can be quantified.

The term “solvent” shall be used to describe any solvent, including water itself or as an aqueous solvent, which may be used to solubilize, disperse or gel a thermoplastic resin or an elastomer which is used in compositions according to the present invention. Exemplary solvents for use in the present invention, in particular, for dispersing, solubilizing and/or gelling the elastomer (which may be thermoset and/or thermoplastic, preferably thermoset), include water, alcohol (especially ethanol, propanol and isopropanol), ketones, isododecane and other hydrocarbon solvents such as isohexadecane, esters, including Dermol 99 (isononyl isononanoate), dimethicone, phenyldimethicone, cyclomethicone (especially D4, D5 and D6, especially D5), low molecular weight dimethicones, among others and mixtures thereof. Solvents which are included in the present invention, may be included for their ability to solubilize a component and/or for their ability to assist in plasticizing the thermoplastic resin in conjunction with the elastomer (quite often a thermoset elastomer) to provide favorable film-forming characteristics according to the invention.

It is believed that in the present invention, a plasticizer, in particular, an elastomer, preferably a thermoset elastomer, functions as a plasticizer and occupies the space between the molecules of the film/thermoplastic resin. Plasticizers are chosen from ingredient classes that are non volatile. Most plasticizers are low molecular weight esters and are generally not particularly useful in the present invention, as these materials tend to volatilize off and make the films not durable enough for commercialization. As low molecular weight esters they tend to stay in the voids created by the cohesion inherent in a film former as the film former solvent begins to evaporate. In typical prior art films, higher molecular weight ingredients are not the plasticizer of choice, rather low molecular weight plasticizers are preferred. However, low molecular weight plasticizers will exude out of the thermoplastic resin over time. Loss of the plasticizer could occur through adsorption onto the substrate. This would reduce the function of the plasticizer and cause the plasticized film to lose flexibility and increase the rigidity of the film causing flaws. Under certain conditions of time, heat, microwave, or choice of plasticizer, exudation will occur, resulting in poor film quality. In one embodiment, the present invention has addressed this difficulty by copolymerizing a plasticizer into or onto the thermoplastic resin or the elastomer, preferably a thermoset silicone elastomer or grafting a plasticizer onto the thermoplastic resin or preferably, a thermoset elastomer. This is to chemically bond the plasticizer into/onto the thermoplastic resin and/or (often) the thermoset elastomer resin and provide stable non-leaching plasticizer characteristics to the final film compositions and films produced therefrom. The final films exhibit favorable characteristics of the plasticizer (rendering the film softer to the touch on keratinous tissue with greater adhesion properties) and as well as greater strength, stability and durability to the final film because of the plasticizer component is chemically bonded in a manner which is integral to the film.

A typical plasticizer (external) is a liquid that is held in place by the thermoplastic elastomer (TPE). By definition, a typical TPE dictates that this liquid exudation is to be expected. In a typical prior art approach, a TPE will soften and lose cohesion due to a lowered glass transition. Adding a plasticizer, further lowers glass transition. Dispersion scanning calorimetry (DSC) analysis can be used to detect a lowering of the glass transition.

This is fundamentally how “plasticizing” is defined (a component that materially lowers the glass transition temperature of a polymer or mixture of polymers to which the plasticizer is added). In contrast, the present invention is directed to a novel approach whereby the elastomer, whether thermoset or thermoplastic provides a non-volatile plasticizer character to the thermoplastic resin, instilling favorable qualities to the films. In addition, in embodiments according to the present invention, a plasticizer component may be provided which is chemically integral (i.e., chemically bonded) within or to the thermoset elastomer. In addition, an external plasticizer may be added to the final composition, including at high weigh rations (i.e. up to about 60% or more by weight of the final film produced from deposition of the composition and evaporation of any solvents included in the composition). In providing for such polymers, a plasticizer is copolymerized with other monomers to produce a thermoplastic resin or thermoset elastomer or alternatively, a plasticizer is grafted onto a thermoplastic resin or a thermoset elastomer to provide additional stable plasticizing characteristics to the thermoset elastomer. In preferred aspects of the present invention, the thermoset elastomer is a thermoset silicone elastomer to which plasticizer components are chemically grafted onto the elastomer. These are described in detail herein.

In the case of the use of a thermoset elastomer in the present invention, the invention utilizes a thermoset elastomer (TSE) which is carrying or is swelled in a solvent. That solvent is, for example, isododecane, isohexadecane, dimethicone, cyclomethicone, isononyl isononanoate, isooctyl isononanoate or other non polar solvent, preferably a solvent which is biologically compatible with and is used in personal care compositions.

If the TSE is in isododecane, the plasticizing thermoset elastomer that is introduced is a plasticizer that may contain a volatile plasticizer (external) component. For example, NuLastic™ ID-LSA 10% (Alzo International, Sayreville, N.J.) contains 10% TSE of a thermoset elastomer and 90% isododecane (volatile plasticizer). If the blend contains a 50% NuLastic™ ID-LSA, the material contains 50% TSE and 50% volatile plasticizer. During and after the film is applied, the film will dry, leaving behind the TSE. The TSE which is left behind is the plasticizer in the final film which is formed. It is a solid, silicone rubber plasticizer. This plasticizer will not exude out of the thermoplastic resin since it is a solid not a liquid. TSE do not exhibit a glass transition under normal conditions, normal conditions being about >20° C., principally because of crosslinking, etc. Glass transitions for TSE's however, can be measured under extreme conditions. They need to be frozen before a glass transition is observed. Thus, in one embodiment, the thermoset elastomers which are useful in the present invention are those thermoset elastomers which exhibit a glass transition when frozen (at a temperature of less than 0° C. and preferably less than about −15° C.).

In certain embodiments, the TSE may contain a volatile solvent, in which case it is included in compositions according to the present invention in higher weight percentages (about 40% to about 80%, about 50% to about 65% by weight of the final film). However, if the TSE does not contain a volatile solvent, but rather a non-volatile solvent which can function as a plasticizer (e.g., isohexadecane, dimethicone, cyclomethicone, isononyl isononanoate, or other non-volatile non-polar solvent), for example, NuLastic™ D-99 LSA which contains the non-volatile Isononyl Isononanoate at 90% and the TSE is at 10%, the amount of TSE included in the film can be included in lower weight percentages, such as 10% to about 50%, about 10% to about 35%, about 10% to about 25%), particular, where the non-volatile solvent functions as a plasticizer to slow down the exudation of solvent and maintain the integrity of the produced film. In stark contrast to the inclusion of a TSE which anchors the non-volatile solvent plasticizer, the use of a non-volatile solvent plasticizer in the absence of TSE, creates a very weak film or no film at all with the film not setting up due to loss of sufficient cohesion, and the dry film exuding the solvent at a rate and a level that interrupts film formation and film adhesion. Thus, the TSE anchors the non-volatile solvent and provides favorable, in most instances, exceptional film-forming characteristics, even when the blend contains extremely high levels of a non-volatile plasticizer solvent. The present invention have produced blends that contain 45% dimethicone or 45% Isononyl Isononanoate or more and have even incorporated 10% fragrance oil (oil soluble and water soluble fragrances) to blends that contain 45% Isononyl Isononanoate or dimethicone and still have achieved film formation. This is an unexpected result. Water based thermoplastic adhesives would not form a film if the thermoplastic contains an external plasticizer at low levels, e.g., 2-4% would plasticize a thermoplastic so that it would not form a dry film. The film would be tacky for a long time and in some cases would never form a film. Surprisingly combining a cross linked thermoset elastomer and a water based thermoplastic elastomer can produce film that contain up to 70% (often 60% or less) of a non-volatile solvent and still form a film without any tack. This film would exude this non-volatile solvent and provide long lasting emolliecncy, a favorable characteristic. Exudation of this non-volatile solvent to the surface of the film will increase the shine of said film. The cross linked thermoset elastomer would be dispersed in a non-volatile solvent and would provide the ability to make formulas containing the thermoset and a high level of non-volatile solvents.

An additional aspect of the invention is emulsification or surfactancy, which includes self-surfactancy or self-emulsifying. The typical plasticizers described in the literature are meant for use in the plastic industry, which are generally anhydrous systems and are not water based or dispersed TPE's. Thus, in certain embodiments of the invention, the inclusion of a non-volatile plasticizer solvent in combination with a TSE and optionally, an additional solvent to solubilize and/or gel the TSE may provide a particularly useful combination, in combination with the thermoplastic resin, but in order to provide the compatibility of the various components to produce films, the inclusion of an external emulsifier or surfactant or a thermoset elastomer exhibiting self-emulsifying characteristics should be included. For example, if an ester or dimethicone plasticizer is to be added to a water based TPE or TSE, the inclusion of an external emulsifier or a TPE or TSE which exhibits self-emulsifying characteristics would need to be added to achieve a homogenous system. The level of surfactancy in a water based TPE or TSE will depend upon the level or amount of hydrophilic groups or chains which are found in the TPE or TSE. The amount of external and/or self-emulsification/surfactancy included in compositions according to the present invention will depend upon the type and amount of solvent. With small amounts of solvent (generally, about 5-10% by weight or less, the amount of emulsifier or self-emulsifier is often reduced), but in many embodiments according to the present invention, there is a substantial amount of solvent added, necessitating a significant amount of emulsifier as otherwise described herein.

Another attribute of a successful emulsion is an increase in viscosity. In certain aspects of the invention, the use of a gelled TSE (TSE gelled in a solvent) is useful for compatibilizing the components to produce a viable film having excellent film characteristics For example, even if one were to emulsify small amounts of esters into a water based TPE, the viscosity increase would often not be sufficient and in most cases would not be appreciable. In certain aspects it is preferred that the TSE is gelled.

The term “emulsifier”, “external emulsifier” or “surfactant” is used throughout the specification to describe a compound which is added to certain compositions according to the present invention in order to compatibilize the aqueous solvent, the thermoplastic resin, the elastomer and the solvent in which the elastomer is solubilized, dispersed or gelled. Emulsifiers as used generally are considered surfactants which exhibit good surface activity and produce a low interfacial tension in the system in which it is used. Mixtures of emulsifiers also may be used, especially where one of the emulsifiers is preferentially oil-soluble and at least one of the emulsifiers is preferentially water-soluble (or dispersible). One of ordinary skill in the art may readily determine the type and amount of emulsifier or emulsifying system (group of emulsifiers) which may be used in the compositions according to the present invention which include water.

Exemplary emulsifiers for use in the present invention may be non-ionic, anionic, cationic or amphoteric and include any cosmetically acceptable emulsifier. Emulsifiers for use in the present invention include, for example, linear or branched chain alcoholic ethoxylates and ethoxysulfates, alcohol ethoxylates, sorbitan esters and ethoxylated polysorbate esters, ethoxylated alkylphenols, for example, polyethoxynonylphenols, phenoxypolyalkoxyalcohols, for example, nonylphenoxypoly(ethyleneoxy)ethanol and nonylphenoxypolyethoxyethanol, hydrophobic or hydrophilic compounds such as ethylene oxide condensation products with higher fatty acids, higher fatty alcohols, or alkylated aromatic hydrocarbons, higher molecular weight poly propylene glycols, amide and amine condensation products of which N-bis(2-hydroxyethyl)-lauramide is exemplary. Other nonionic emulsifiers may include polyoxyethylene ethers including polyoxyethylene isohexadecyl ether, such as Arlasolve™ 200 (available from ICI Americas), polyoxyethylene lauryl ether such as Brij 35™, polyoxyethylene stearyl ether, for example Brij 72™ and Brij 78™ and polyoxypropylenestearyl ether, among others. Other exemplary emulsifiers include ethoxylated lanolin, for example, Lanogel 41 (Lubrizol, Inc. Cleveland, Ohio), alkyl and dialkyl succinate compounds, including combinations of these emulsifiers.

Exemplary anionic emulsifiers for use in the present invention include, for example, soaps, such as triethanolamine stearate, alkaline salts of sulfuric acid esters of polyhydric alcohols, e.g. sodium lauryl sulfate, sodium cetyl sulfate, etc., higher fatty sodium alcohol sulfates, such as those derived from coconut oil, hydroxyl sulfonated higher fatty acid esters such as fatty acid esters of 2,3-dihydropropane sulfonic acid, high fatty acid esters of low molecular weight alkylol sulfonic acids, e.g., the sodium oleic acid ester of isethionic acid, sulfated higher fatty acid alkylolamides such as ethanol amide sulfates, higher fatty acid amides of amine alkyl sulfonic acids, such as lauric amide of taurine, among others and armomatic containing anionic synthetic emulsifiers. Exemplary amphoteric emulsifiers include, for example, salts of N-alkyl compounds of betaamino propionic acid wherein the alkyl group is derived from a fatty acid such as a mixture of coconut oil fatty acids, among others. Exemplary cationic surfactants include ammonium and quaternary salts of fatty amines and substituted fatty amines, among others. All of the above emulsifiers, among numerous others, may be used alone or in combination with other emulsifiers to make compositions according to the present invention. It is noted that when using a cationic emulsifier, caution must be maintained in selecting a thickener for use in the present invention.

Other emulsifiers for use in the present invention include propylene glycol-isoceteth-3-acetate (Hetester® PHA, available from Alzo International, Inc., Sayreville, N.J., USA), self-smulsifying glyceryl monostearate cetearyl alcohol, ceteareth-20 and mixtures, thereof.

The term “oil” is used throughout the specification to describe any of various lubricious, hydrophobic and combustible substances obtained from animal, vegetable and mineral matter, but preferably are derived from vegetable (i.e., non-animal, “green” sources) which may be included to embellish certain compositions or lower the cost of certain compositions according to the present invention. Emollient oils for use in the present invention may include petroleum-based oil derivatives such as purified petrolatum and mineral oil. “Non-polar” oils are generally oils such as petrolatum or mineral oil or its derivatives which are hydrocarbons and are more hydrophobic and lipophilic compared to synthetic oils, such as esters, which may be referred to as “polar” oils. It is understood that within the class of oils, the use of the terms “non-polar” and “polar” are relative within this very hydrophobic and lipophilic class, and all of the oils tend to be much more hydrophobic and lipophilic than the water phase which is used to produce the water-in-oil and oil-in-water emulsions as well as Non-dispersible (floating) bath oils of the present invention. Preferred hydrophobic oils for use in the present invention include mineral oil and petrolatum. Preferred less hydrophobic (i.e., more polar) oils for use in the present invention include a number of maleates, neopentanoates, neopentanoyls, citrates and fumarates, and any other cosmetically acceptable ester emollient. In the case of Non-dispersible (floating) bath oils, preferred emollient oils other than the capryl isostearate include mineral oil and vegetable oil.

Additional oils for use in the present invention may include, for example, mono-, di- and tri-glycerides which may be natural or synthetic (derived from esterification of glycerol and at least one organic acid, saturated or unsaturated, such as for example, butyric, caproic, palmitic, stearic, oleic, linoleic or linolenic acids, among numerous others, preferably a fatty organic acid, comprising between 8 and 26 carbon atoms). Glyceride esters for use in the present invention include vegetable oils derived chiefly from seeds or nuts and include drying oils, for example, linseed, iticica and tung, among others; semi-drying oils, for example, soybean, sunflower, safflower and cottonseed oil; non-drying oils, for example castor and coconut oil; and other oils, such as those used in soap, for example palm oil. Hydrogenated vegetable oils also may be used in the present invention. Animal oils are also contemplated for use as glyceride esters and include, for example, fats such as tallow, lard and stearin and liquid fats, such as fish oils, fish-liver oils and other animal oils, including sperm oil, among numerous others, but these are less preferred. In addition, a number of other oils may be used, including C₁₂ to C₃₀ (or higher) fatty esters (other than the glyceride esters, which are described above) or any other acceptable cosmetic emollient.

The term “polybutadiene” shall mean, within the context of its use, a polymeric material which is produced from butadiene monomers. Polybutadiene polymers for use in the present invention have a structure according to the chemical formula:

CH₃—HC═CH—CH₂CH₂—HC═CH—CH₂ⱼCH₂—HC═CH—CH₃

Where j is from 5 to about 500 or more, about 16 to about 200; about 30 to about 150, about 40 to about 100, about 90-110, about 100. Preferred polybutadiene polymers for use as multifunctional hydrocarbon polymers for incorporation into a silicone thermoset polymer for use in the present invention comprise about 5% to about 50% by weight of olefinic character (also referred to as “vinyl content”—based upon the molecular weight of olefin within the polybutadiene molecule), about 5 to about 35% by weight olefin, about 15% to about 25% by weight olefin. Preferred polybutadiene polymers for use in the present invention comprise about 90+% cis olefins (of a mixture of cis and trans olefins within the polybutadiene molecule), about 95+% cis olefins, about 99+% cis olefins, about 99.5+% cis olefins, about 99.9+% cis olefins. It is noted that the polybutadiene component of the present invention contains a number of vinyl groups which may react with Si—H or other groups (as otherwise described herein) within an active silicone elastomer precursor to produce silicone thermoset polymers elastomers according to the present invention. In the present invention, it is contemplated that the multifunctional hydrocarbon (polybutadiene), especially including polybutadiene functions as a hydrocarbon backbone in the silicone thermoset elastomer polymers according to the present invention.

Preferred polybutadiene polymers for use in the present invention comprise about 0.005% to about 7.5% by weight of the final silicone crosslinked hydrocarbon elastomer, about 0.05% to about 5% by weight, about 0.1% to about 2.5% by weight, about 0.25 to about 4%.

Because of the physicochemical characteristics of polybutadiene and its ability to react with hydrosilane terminated polydimethylsiloxanes, the compatibility of polybutadiene as a reactant with silicone crosslinking agents/chain extenders as otherwise described herein results in final compositions which can be manufactured with a high degree of purity, consistency, gelation characteristics, flexibility and compatibility for inclusion in personal care products along with thermoplastic resins as described herein. It is noted that the inherent high compatibility between the polybutadiene polymer backbone and the silicone crosslinkers/chain extenders (of varying compositions as otherwise described herein) provides an easily and consistently manufactured silicone elastomer which can be varied quite markedly in final characteristics by incorporation of additional components (such as allyl alcohol ethoxylate and polyurethanes) as otherwise described herein. The polybutadiene instills further “rubber-like” feel and flexibility to the final films, which is advantageous in certain films, depending on their end-use.

The term “polyurethane” shall mean, within the context of its use, a polymeric urethane compound comprising at least one and preferably, two or more urethane linkages which are generally formed by reacting at least one compound containing a free alcohol (primary, secondary or tertiary), preferably at least one compound containing at least two alcohol groups (“polyol”) and a diisocyanate compound. Thus, the term polyurethane as used herein incorporates dimer urethanes (those compounds which contain two urethane bonds) which are formed from a diisocyanate and a monohydric alcohol of varying structure, which structure may contain, for example, an active group or a protected active group such as a silyl-protected hydroxyl group or amine group wherein the protecting group may be removed subsequent to formation of the polyurethane or an olefinic group (such as for example, a vinyl group, acrylate or methacrylate group) which can participate in a reaction with a silane group from the silicone polymer crosslinker/chain extender to produce a silicone elastomer having a hydrophilic compound and self-emulsifying characteristics for use in the present compositions.

In addition, polyurethanes according to the present invention preferably are formed by reacting at least one polyol (a compound which is either hydrocarbon or siloxane based and which contains at least two free hydroxy groups with a diisocyanate to produce a polyurethane, with the polyol optionally and preferably containing at least one functional group which does not participate in the polymerization reaction to form the polyurethane composition, but which, subsequent to the polymerization reaction, can be used to crosslink the polyurethane composition to a silicone elastomer in certain embodiments according to the present invention. In preferred aspects of the invention, polyurethane compounds which are reacted with a silicone elastomer to produce hydrophilic silicone elastomers preferably have sufficient hydrophilic character (for example, by containing sufficient hydroxyl groups and/or ethoxylated-polyethylene oxide or PEG groups) to instill hydrophilic/self-emulsifying character to the final hydrophilic silicone elastomers which may be used in the present invention.

In certain embodiments, the silicone elastomer incorporates urethane polymers according to the present invention have the general structure Formula V:

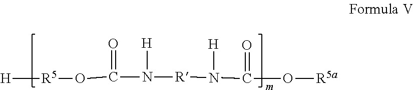

Where R⁵ is an optionally substituted hydrocarbon or optionally substituted siloxane group, preferably, an optionally substituted (with hydroxyl groups and/or PEG groups comprising from 1 to 100 or 2 to 25 ethylene oxide units) C₁-C₅₀ hydrocarbon group containing at least one olefinic group or a polyethylene oxide group comprising between 1 and 500, 2 and 100, 5 and 25, 5 and 20, 5 and 15 ethylene oxide groups which may be optionally endcapped with or contain a polymerizable group such as an alkenyl or (meth)acrylate group, or
a siloxane group according to the structure:

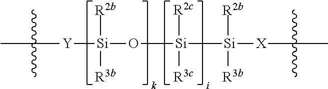

and
R^{5a} is an optionally substituted hydrocarbon (which may contain hydroxyl and/or PEG groups as otherwise described here) or a siloxane group, preferably, an optionally substituted C₁-C₅₀ hydrocarbon group, optionally containing at least one olefinic group, or a siloxane group according to the structure:

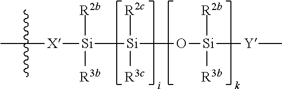

Wherein Y is absent, O or a

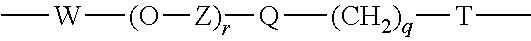

group;
X is absent or a

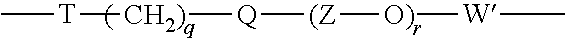

group;
X′ is absent or a

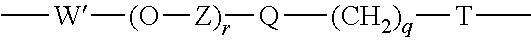

group;
Y′ is absent or a

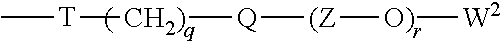

group;
W is absent when r is an integer of 1 or more and W is absent or O when r is 0;
Q is absent or 0;
q is an integer from 0 to 10, preferably 1 to 6, preferably 1 to 3;
r is an integer from 0 to 100, 0 to 40, preferably 1 to 20 or 1 to 10, with the proviso that q or r is at least 1;
T is absent or O;
W′ is absent when r is 0 and is a Z group when r is 1 or more;

### W² is H;

Z is independently an ethylene group, a propylene group or a mixture of ethylene and propylene groups;
R^{2b} and R^{3b} are each independently a C₁-C₁₀ alkyl group (preferably both are a C₁-C₃ alkyl group, preferably both are methyl groups), preferably R^{2b} and R^{3b} are both C₁-C₁₀ alkyl groups, preferably C₁-C₃ alkyl groups, preferably both are the same C₁-C₃ alkyl group, preferably both are methyl groups;
R^{2c} and R^{3c} are independently selected from an optionally substituted C₁-C₆ alkyl group (substitution with OH or a C₁-C₃ alkyl group which itself may be optionally substituted with a hydroxyl group) and optionally, an Si—H group, an alkenyl group and/or a hydroxyl group in small percentages of the total number of R^{2c} and R^{3c} substituents within the polymer. In certain embodiments, R^{2b}, R^{3b} R^{2c} and R^{3c} optionally may comprise a small percentage (i.e., less than about 2%, 1.5%, 1.0%, 0.75%, 0.5%, 0.25%, 0.1%, 0.05% or 0.002%) of Si—H groups, alkenyl groups and/or hydroxyl groups of the total number of R^{2b}, R^{3b} R^{2c} and R^{3c} groups which are found in the silicone group;
R′ is an optionally substituted C₂ through C₃₆ (preferably, C₆ through C₂₂, most preferably an isophorone group) linear, cyclic or branch-chained saturated or unsaturated hydrocarbon group (which may be monomeric or dimeric, an aromatic group, including a phenyl or benzyl group or substituted phenyl or benzyl group, an alkylphenyl, alkylbenzyl or substituted alkylphenyl or alkylbenzyl group);
i is an integer from 0 to 50, preferably 0, 1, 2, 3, 4, 5, 6, 7, 8, 9 or 10 (preferably 0 or 1);
k is an integer from 0 to 100, 1 to 100, about 5 to 50, about 10 to 45, preferably about 20 to 40; and
m is from 1 to 100,000, about 1 to 25,000, about 5 to 25,000, about 50 to 20,000, about 50 to 20,000, about 100 to 20,000, about 100 to 10,000, about 200 to 5,000, about 250 to 2,500, about 500 to about 2,000, 1 to about 1,000, 1 to about 750, 2 to about 650, about 50 to 15,000, about 10 to 10,000, about 200 to 5,000, about 250 to about 2,500, about 5 to about 150, about 3 to 100, about 5 to 250. Preferably, the polyurethane according to the present invention is obtained by reacting a polyol (which may be hydrocarbon based or siloxane based and contains at least two hydroxyl groups) with a diisocyanate compound to produce a polyurethane composition accordingly.

In certain preferred aspects of the present invention in the polyurethane formula V described above, R⁵ is a O—R⁶ group and R^{5a} is a R^{6a}—OH group where R⁶ and R^{6a} are each independently an optionally substituted hydrocarbon or an optionally substituted siloxane group as set forth for R⁵ and R^{5a}, respectively and generally described above.

One or more polyols and/or diisocyanates may be used to produce polyurethane polymers according to the present invention, with preferred polyols having, in addition to having at least two free hydroxy groups to participate in polymerization reactions to form polyurethanes, at least one reactive alkene (unsaturated hydrocarbon) group must be available for reaction with the hydrosilane terminated polydimethylsiloxane crosslinking agents of the present invention, and with the diisocyanate preferably being isophorone diisocyanate. Further preferred polyols contain multiple hydroxyl groups or alternatively, polyethylene oxide groups wherein the PEG groups contain from 2 to 100 ethylene oxide groups, preferably 3 to 50, 5 to 25 or 5 to 10.

Alternative polyurethanes according to the present invention also are prepared from a diisocyanate, preferably isophorone diisocyanate, glycerin and glycerin esters, propylene glycol and its esters, dipropylene glycol and its esters, alkyl amines, ethoxylated alkyl amines, propoxylated alkyl amines, silicone ethoxylates and silicone propoxylates, among others.

Certain preferred polyurethanes which may be reacted with silicone elastomer precursors (generally, a crosslinker or a chain-extender, but also including any reactive silicone compound as otherwise described herein) to produce polyurethane-containing hydrophilic silicone elastomers for inclusion in the present invention include, for example, Polyderm PPI-CO (Castor oil/IPDI copolymer), which is obtained by reacting castor oil with isophororone diisocyanate; Polyderm PPI-DGDIS (Polyglycerol-2-Diisostearate/IPDI copolymer); Polyderm PPI-G7-CA (Gycereth-7/Polyglyceryl-2/PEG-15 Cocamine/IPDI Copolymer); Polyderm PPI-GRC (Glycerol Diricinoleate/IPDI Copolymer; Polyderm PPI-PGR (Propylene Glycol Diricinoleate/IPDI Copolymer); Polyderm PPI-SA-15 (PEG-15 Soyamine/IPDI Copolymer); Polyderm PPI-SA (Di-PEG-2 Soyamine/IPDI Copolymer); and Polyderm PPI-SI-L (BIS PEG-1 Dimethicone/IPDI Copolymer); all available from Alzo International, Sayreville, N.J. Each of these polyurethane compounds and silicone elastomers prepared therefrom are disclosed in PCT/US2008/010681, published as WO 2009/035676 on Mar. 19, 2009, the entire contents of which is incorporated by reference herein.

The term “secondary plasticizer” or “external plasticizer” is used to describe an external plasticizer, which is optionally included in compositions according to the present invention to enhance or complement the plasticizer characteristics of the elastomer (preferably a silicone elastomer) which is included in compositions according to the present invention. Secondary plasticizers, when they are included in compositions according to the present invention, comprise about 0.01% up to about 80%, often about 0.5% to about 60%, often about 0.75% to about 50%, about 1% to about 50% about 50% to about 80%, about by weight of the final polymer composition (which contains thermoplastic resin, elastomer, solvent and optional external emulsifier). In other embodiments, the external plasticizer comprises about 0.1% to about 7.5% by weight of the final composition, about 0.25% to about 6%, about 0.5% to about 5%, about 0.75% to about 4.5%, about 1% to about 3% of the final composition. It is noted that the external plasticizer may be included in large amounts in the thermoset elastomer composition and small amounts, if at all, in the thermoplastic resin composition. Final films (after deposition onto a surface and evaporation of solvent) may contain upwards of about 60% by weight of an external plasticizer, often between about 0.05% to about 60%, about 0.25% to about 50%, about 0.5% to about 15%, about 1% to about 10%, about 0.75% to about 20%, about 1.5% to about 40%, about 30-60% by weight of an external plasticizer, about 0.1% to about 7.5% by weight, about 0.25% to about 6%, about 0.5% to about 5%, about 0.75% to about 4.5%, about 1% to about 3% of the deposited film. Typical secondary plasticizers are known in the art and include, for example, esters, ethers, waxes, oils, hydrocarbon polymers, among numerous others. Most often, the external secondary emulsifier is an ester. Exemplary secondary ester plasticizers for use in the present invention include, for example, sebacates, adipates, gluterates, phthalates, azelates, and other specialty blends.

Without being limited by way of theory, it is believed that in the present invention, a combination of two rather distinct polymers, i.e. a thermoplastic resin and an elastomer, preferably a thermoset silicone elastomer, produce an integral film because of the ability of the films to be compatible such that unexpected film-forming characteristics are realized. In one embodiment, the present invention relates to the use of silicone elastomers as a plasticizer in water based or water dispersed thermoplastic resins, which optionally contains an external emulsifier and further optionally, a secondary external plasticizer. The components are combined and deposited as a film on a surface producing a water resistant film have unexpected properties associated with wear resistance and transfer resistance. In the present invention, the use of silicone elastomers as a plasticizer in a water based or water dispersed thermoplastic resin, which optionally contains an emulsifier, provides compositions, which, when deposited on surfaces produce a film of unexpectedly favorable characteristics of water resistance, wear resistance and transfer resistance, among others. Without being limited by way of theory, it is believed that these elastomers function as plasticizers imparting flexibility in a film by creating spaces between the molecules of the thermoplastic resin which appear to reduce the glass transition of the polymer, softening the polymer and reducing the viscosity of the adhesive (thermoplastic resin). This aids in the diffusion of the polymer onto and into the substrate, and further integrates the elastomer into the structure of the film. Diffusion of the thermoplastic resin (which is water dispersible) onto the substrate creates better adhesion due to its increased contact or wetting of the surface, and rather unexpectedly, the elastomer integrates with the thermoplastic resin to provide an integral film with unexpectedly superior film characteristics. Polar ingredients will reduce the viscosity of cross linked silicone thermoset elastomers. All water borne thermoplastic elastomers are polar. This polarity allows for the thermoplastic elastomer to mix/combine homogenously throughout the 3 dimensional structure of a cross linked thermoset elastomer. Reducing the viscosity of the cross linked silicone thermoset elastomer a better emulsion is formed. The present invention can thus be defined as a homogenous blend of the two ingredients thermoplastic and thermoset. This homogenous emulsion will yield a homogenous film of thermoplastic and thermoset.

A monomolecular thermoplastic resin or adhesive (i.e., a polymer formed from the polymerization of a single monomer) produces the strongest bond and, depending on end-use, may be preferred for use in the present application. This mono-molecular thermoplastic resin must have sufficient adhesion and just as critically, it must be cohesive. Cohesiveness of the polymer means that as the water and or solvent evaporates, the molecules in the resin will come into contact and join together to form a continuous film. Non-elastomeric plasticizers can cause a reduction in the cohesiveness of the film since the plasticizer will cause separation of the adhesives molecules, a negative result, and typically, the plasticizer will increase the likelihood that a film produced therefrom will exhibit weakness and fail because of free spaces or voids in the film which occur. In addition, any voids or flaws in the film reduces cohesion and therefore reduces contact with the substrate to which the film is applied to.

While there are many things that can contribute to the formation of micro flaws in a thermoplastic resin, other factors include the composition of the thermoplastic resin, its viscosity, the condition of the substrate, evaporation rate of the solvents included in the composition and the inclusion of other ingredients in the final composition, may all contribute to the formation of micro flaws. Indeed, virtually any ingredient that becomes part of the adhesive film can reduce cohesion and adhesion. However, the inventors of the present invention have discovered that the inclusion of a thermoset elastomer, preferably a thermoset silicone elastomer, in effect amounts can reduce and/or eliminate these micro flaws and produce a uniform, continuous film. Thus, one of ordinary skill may readily adjust compositions according to the present invention and enhance various features and characteristics of the thermoplastic resin by simply fine-tuning the amount and type of elastomer, especially a thermoset silicone elastomer in compositions according to the present invention.

The present invention utilizes silicone elastomers to plasticize thermoplastic resin adhesives, which produces a superior continuous film with unexpected characteristics. Silicone elastomers are used in cosmetic compositions for various functions. Elastomers and specifically silicone elastomer are not commonly used as plasticizers for a water based or water dispersed thermoplastic resin. This invention combines a silicone elastomer with a water based thermoplastic resin. A film created by this compositional blend will create a continuous film. This film can now be peeled or lifted off the substrate in one piece. The elastomer is now part of or integral to the thermoplastic resin. A cast film of a silicone elastomer and the resin will create a uniform, flexible film which is also water resistant. This shows not only a plasticizing effect of the elastomer on the thermoplastic resin but a reinforcement of the thermoplastic resin by the silicone elastomer to produce a continuous, uniform film Rather unexpectedly, the inclusion of the water based thermoplastic resin is filling the voids of the cross linked elastomer and is creating a hybrid film that exhibits substantially better qualities when used together than if used individually.

In certain embodiments, the present invention relates preferably but not exclusively to a silicone elastomer that's been hydrophilically functionalized. U.S. Pat. No. 6,936,686 teaches the use of alkylene ethoxylate as a co-reactant to increase the hydrophilicity of the resulting elastomer. Such hydrophilic elastomers find great utility as water in oil (W/O) emulsifiers, as protectants and carriers for other water soluble ingredients and as fully functional silicone elastomers having the same expected properties previously described. In addition to alkylene ethoxylate, the silicone elastomers used in the present invention may also include units obtained from allyl alcohol ethoxylate and/or various hydrophilic polyurethanes, which also provide hydrophilic components in the present invention.

Compositions may also include hydrophilic silicone elastomers prepared from an admixture of a traditional hydrophobic silicone elastomer with a hydrophilic polyurethane or the chemical reaction product of a silicone elastomer with an alkene containing hydrophilic polyurethane. These hydrophilic polyurethanes silicone elastomers provide additional solubility opportunities (because the polyurethane solubility characteristics can be widely varied i.e. through increased/reduced hydrocarbon solubility), controllable hydrophilicity and variable deposition capacity (because the polyurethane can optionally contain amine or quaternary ammonium salt functionalities to facilitate the deposition of the polymer on surfaces). Additionally, an amine moiety helps to disperse pigments in compositions that are typically nonionic. Amine functionality also is typically known to promote good adhesion on surfaces that are anionic such as glass, plastic, and on skin and hair.

Amines interact with water. Primary, secondary and tertiary amines all are water soluble. Primary amines are the most water soluble while tertiary amines are less water soluble. The water solubility or hydrophilicity of a tertiary amine is reduced by its hydrophobic moiety. Thus by modifying the amine used in the silicone elastomer, this can aid in dispersing pigments in non-ionic emulsions. Thus, the inclusion of an amine or ammonium salt functionality modifies an otherwise neutral, non-polar silicone elastomer into silicone elastomer having polar, cationic character. A silicone elastomer which contains amine functionality can have a neutralizing effect on an acidic pH.

A multifunctional silicone elastomer is an optionally used silicone elastomer for use in the present invention. A silicone elastomer that has film forming properties, emulsifying properties, enhanced solubility of both hydrophilic and lipophillic properties and has a cationic charge is an example of a multifunctional silicone elastomer. Quite unexpectedly, these attributes are ideal for blending water based thermoplastic resins and achieving a compositional result that is far superior to past attempts at creating long lasting, water and transfer resistant films. The multifunctional elastomer can be used to emulsify solvents and thermoplastic resin, aid in pigment dispersion, improve film forming properties, increase compatibility with hydrocarbons and/or silicone type ingredients, and can play a role in controlling the pH.

Typical silicone elastomers that have not been hydrophilically modified or do not contain amine moieties can nonetheless plasticisze water based thermoplastic resins with great efficiency. However, a multifunctional silicone elastomer as previously defined would be soluble in hydrocarbon and silicone based ingredients, exhibit hydrophilicity, and carries a cationic charge, thus providing additional attributes to the final film composition. A silicone elastomer combined with an emulsifier would enable water based materials to be used in emulsions to the exclusion of non-aqueous solvents, an advantage in numerous applications, in particular personal care compositions. Both the elastomer and the emulsifier would have to be soluble and or compatible with thermoplastic resins. Compatibility would ensure that a homogenous film formation would be made. This is critical in maintaining proper adhesion and just as important, cohesion would be maintained. A cationic ingredient such as amodimethicone can be used to aid in dispersing pigments. The cationic ingredient can also aid in pH control. Compatibility may be increased by “dialing-in” or modifying the silicone elastomer composition as otherwise described herein or improving the compatability of the polymers and solvents through inclusion of an external emulsifier, with greater quantities of the emulsifier utilized where required to emulsify the thermoplastic resin and the elastomer. One of ordinary skill will readily be able to modify the type and amount of external emulsifier to emulsify the polymers to produce films of exceptional film forming characteristics.

Water based film formers (thermoplastic polymers) are used to create long lasting, transfer resistant, and water resistant films. These include, for example, acrylonitrile butadiene styrene polymers, polyacrylic or poly(meth)acrylic resins, celluloid cellulose acetate, cyclic olefin copolymers, ethylene-vinyl acetate, ethylene vinyl alcohol, a fluoroplastic, acrylic/polyvinylchloride copolymer, liquid crystal polymer, polyacrylonitrile, polyoxymethylene, polyamide (nylon), polycarbonate, polyamide-imide, polyaryletherketone, polybutadiene, polybutadiene/styrene copolymers, polybutadiene/acrylic copolymers, polybutadiene/acrylamide copolymers, polybutylene, polybutylene terephthalate, polycaprolactone, polychlorotrifluoroethylene, polyethylene terephthalate, polyhydroxyalkanoates, polyketone, polyester, polyethylene (both low and high density), polyetheretherketone, polyetherketoneketone, polyaryletherketone, polyetherimide, polyethersulfone, chlorinated polyethylene, polyimide, polylactic acid, polymethylpentene, polyphenylene, polyphenylene oxide, polyphenylene sulfide, polyphthalamide, polypropylene, polystryrene, polysulfone, polytrimethylene terephthalate, polyurethane, polyvinyl acetate, polyvinyl chloride, polyvinylidene chloride and styrene-acrylonitrile, among others.

These water based film formers are often used as adhesives and have adhesive-like characteristics. When applied to a surface such as skin, hair and nails of a patient or subject, the thermoplastic resin functions as a film former. In addition, the thermoplastic resin advantageously dries to a film which is capable of delivering typical cosmetic ingredients or bioactive agents onto the skin, hair or nails of a patient or subject. Compositions may be sufficiently viscous and can be readily formulated using various personal care components so that solids such as colorants would not settle to the bottom of the package. Most water dispersible thermoplastic resins tend to be low in viscosity. There are three ways to increase the viscosity of these materials. One is to use a thickener as an additional component. The second way is to use an emulsifier to create an emulsion. The third way is to use both a water thickener and emulsifier. All three methods increase the ability of the thermoplastic resin to limit or eliminate the settling of colorant (dyes/pigments) components. Adding a thickener, if required, reduces the water solubility and increases the water resistance, transfer resistance, and adhesive qualities associated with thermoplastic resins. U.S. Patent Application Publication No. 20100260687, incorporated by reference in its entirety herein, describes the use of an aqueous polyurethane dispersion in cosmetic compositions in this regard.

In certain embodiments, the present invention also focuses on personal care applications and specifically compositions that deliver actives, colorants, and ingredients commonly used in the personal care industry onto the surface of the skin, nail, or hair. These ingredients can be water, an aqueous solvent (e.g. alcohol or other compatible solvent), a non-aqueous solvent, emollients, humectants, oils (polar and non-polar) conditioning agents, surfactants/emulsifiers, thickeners/thickening agents, stiffening agents, emulsifiers, medicaments, fragrances, preservatives, deodorant components, anti-perspirant compounds, skin protecting agents, pigments, dyes, coloring agents, sunscreens, waxes, sunscreens, AP-DEO ingredients, clays and minerals, etc. and mixtures thereof, among others.

The compositions according to the present invention are used to maintain other ingredients on a substrate over an extended period of time. In the case of products in the lip category (e.g., lipsticks and lipglosses), the typical prior art product wears about 2 hours. The use of the compositions according to the present invention provides that colorants stay on the applied area longer than the typical 2 hours and in some cases the period of wear can be as long as 24 hours or longer. The lips are a particularly difficult substrate to adhere product to. The lip, compared to the face or hair is a substrate that is in constant movement and under continuous interaction with itself and its environment. Also, the lips have two surfaces, upper lips and bottom lip to which a product is applied. These two substrates are in constant motion. They are continuously in contact with each other. There is contact with saliva as well as contact with liquids or food. These liquids and solids come into contact with the lips at varying temperatures. This makes development of long wear products very difficult.

Most water based resins do not provide ideal film formation. Thermoplastic resins can function as a tackifier, enhancing adhesion (i.e., they increase adhesion because of an increase in tack, which could compromise transfer resistance). Since they are water based resins they tend to be soluble or dispersed in water and therefore provide films that are often limited in their water resistance, or transfer resistance in varying degrees. In prior art applications, resins that are not as water resistant are used as tackifiers (they can also function as binders). The term tack is often synonymously defined as adhesion. The resin providing adhesion to a surface is often sticky or tacky. It also has cohesion, but it sticks to itself. Cohesion also serves the purpose of binding all ingredients into a final film such as mascaras. However, in the present invention, the films which are formed are often tack-free and consequently, provide cohesion to a surface, as well as a tack-free surface which exhibits exceptional transfer resistant properties.

Water based thermoplastic resin dispersion can contain about 5% to 80% by weight resin in solution, but often about 30-40%. A personal care composition can contain about 5-60%, about 10-50%, about 5-40%, about 10-45%, about 5-35% by weight of this aqueous dispersion along with the other components. The level of thermoplastic resin used can increase adhesion, and to some extent, water resistance, but increasing the aqueous dispersion will increase the difficulties of formulating with these resins. Since the resin is dispersed in water one needs to formulate an emulsion or use water thickening ingredients. The emulsifiers and or water thickening ingredients increase water solubility and consequently compromise water resistance and to some extent, durability or wear resistance of the film.

The present invention teaches that by mixing a hydrophilically modified or self emulsifying silicone elastomer and thermoplastic resin dispersion one can achieve better film forming properties, which can accommodate a large number of components without compromising film characteristics, especially if greater wear and water resistance is an important characteristic for the desired films. Self emulsifying silicone elastomers as otherwise described herein will emulsify the water based thermoplastic resin without increasing the water solubility of the film. The self emulsifying silicone elastomer increases the viscosity of the water based thermoplastic resin by emulsifying the thermoplastic resin. Conventional plasticizers function by keeping the resin molecules apart. Silicone elastomers surprisingly do the same and reinforces, modifies, or alters the dry films attributes.

Not only do the silicone elastomers, plasticize the thermoplastic resin but do so without jeopardizing the film forming properties of the thermo plastic resin. One embodiment combines NuLastic™ Silk-E-D99-LSA by Alzo International INC with Polyderm PE/PA, also made by Alzo International Inc. Polyderm PE/PA is water based thermoplastic resin. The film formed using this resin, while quite good, is not nearly as excellent as a film produced where Polyderm PPG-20, replaces Polyderm PE/PA. The film produced using NuLastic™ Silk-E ID LSA (multifunctional thermoset silicone elastomer) with Polyderm PPG-20 exhibits great adhesion, great transfer resistance but will come off with some effort in cold water. The blend of NuLastic™ Silk E ID-LSA (Isododecane, (and) Polysilicone 23 plus Polyderm PPI-PE/PA (Polyether-Propionic Acid/TMXDI Copolymer) results in increased water resistance.

Preferred components used in the present invention include the following commercially available products, which are obtained as reaction products of the following components which are listed below:

### Thermoset Silicone Elastomers

NuLastic™ Silk-E-ID LSA
Isododecane
Vinyl terminated Polydimethyl Siloxane
Dimethyl Methyl Hydrogen Polysiloxane Copolymer
PEG-2 Dimethicone
IPDI (3-Isocyanate Methyl-3,5,5-Trimethylcyclohexyl Isocyanate)
PEG-2 Soyamine
NuLastic™ Surfa D-99
Isononyl Isononanoate
Vinyl terminated Polydimethyl Siloxane
Dimethyl Methyl Hydrogen Polysiloxane Copolymer
Allyl Alcohol Ethoxylate PEG10-15
NuLastic™ Silk MA,DM
   Dimethicone
   C4-C24 Alpha Olefin
   Methyl Hydrogen Dimethyl Dimethicone Copolymer
   Bis-Vinyl Dimethicone
Hydrocarbon Silicone Hybrid Elastomer (NuLastic™ hydrocarbon elastomer)
   Polybutadiene
   Methyl Hydrogen Dimethyl Dimethicone
      Water based TPE's (Thermplastic Resins)

### Polyderm PPG-20

Dimethylol Propionic Acid
POLYDERM PPG-20 (PPG-2-PPG-40 is possible)
IPDI
Triethylamide

### Polyderm PPI PE/PA

Dimethylol Propionic Acid
TMXDI
DMAMP-80
Poltetrahydrofuran

The present invention is now further described using the following examples. These examples should be taken as simply a means to enhance an understanding of the present invention and should not be taken to limit the invention in any way.

### Examples

In the following examples, transfer resistance was measured using the following procedure. This procedure can be used for applications that contain colorants.
1) Sample is applied to the skin, lips, hair, or nails. Application areas will be referred to as the “substrate”.
2) A dry time or set time is established. A 3 minute dry time is typical for transfer resistant products.
3) Substrate that has product applied on it can be pressed against a white surface. White surface can be paper, fabric, smooth white ceramic tiles, etc.
4) Transfer of product from substrate to white surface is determined visually.
5) Transfer of product from substrate to white surface can be quantified by using the following procedure.
   a. Weigh product+ package before applying.
   b. Apply product.
   c. Weigh the product+ package.
   d. Wait three minutes so that the applied film can dry or set.
   e. Weigh the white surface.
   f. Press the coated substrate to the white surface.
   g. Hold for 10 seconds
   h. Remove white surface and weigh.

Calculations: % Product Transferred=(W_{A}−W_{I÷}(P_{I}−P_{A})×100
W_{A}=White surface after transfer
W_{I}=White surface before transfer
P_{I}=Product+ Package before applied
P_{A}=Product+ Package after applied
   Water Resistance is tested using the following method.
1) Product is applied onto a substrate.
2) Substrate can be glass, plastic, fabric, skin, hair, nail
3) Substrate with the applied product is weighed or the package is weighed before and after product is applied.
4) Product is allowed to dry on the substrate
5) After the allotted dry time the substrate is immersed in water and kept there for a given time period.
6) Immersed area is taken out of the water.
7) Product applied to glass or plastic can be weighed for any loss of product.
8) Product applied on the skin can be analyzed by using the Transfer resistance procedure step 5.

Calculations: % Water Resistance=(W_{A}−W_{I÷}(P_{I}−P_{A})×100
W_{A}=White surface after transfer
W_{I}=White surface before transfer
P_{I}=Product+ Package before applied
P_{A}=Product+ Package after applied

### Lip Product

A preferred compositional embodiment of this invention is a two component long lasting lip product. A two component lip product is comprised of an under coat and a top coat. The under coat can contain the following or combinations of the following, a solvent, film former, emollients, waxes, colorants, fillers, suspending agents and can contain typical ingredients used by formulators. This undercoat delivers a makeup effect onto the lips that can be formulated to last from 4-24 hours of wear. These products are transfer resistant, water resistant, and will resist oils. Oil resistance is an attribute that helps the product last on the lips.

The topcoat is comprised of waxes and oils. Oils can be selected from commonly used cosmetic ingredients such as esters, natural oils, polymers. This topcoat can be in the form of a lip stick or a lip gloss. This topcoat provides a shiny non drying emollient coat over the undercoat. This makes for a comfortable product while increasing the protection of the undercoat from water.

A challenge posed by a two component product arises when the topcoat softens the undercoat causing a decrease in wear properties or removal of the undercoat. The ingredients used in the topcoat cannot dissolve the ingredients used in the undercoat. To avoid this issue the ingredients in the topcoat must be incompatible with the ingredients in the undercoat. Using polar oils in one coat and non polar oils in another coat is a common practice. The present invention utilizes a water based thermoplastic resin that is dispersed in water at an alkaline pH (Polyderm PPI-PE/PA has pH of 8-10). The thermoplastic resin reinforced silicone elastomer composition claimed in this patent result in an applied film that will not soften when a topcoat is applied. The combination of the silicone elastomer and the water based thermoplastic resin creates a film that will not soften when in contact with common ingredients used to make a topcoat. The cross linked silicone elastomers are typically removed with solvents since they are soluble in these same solvents. The inclusion of the water borne thermoplastic, specifically water borne polyurethane thermoplastic which is resistant to most organic solvents assists in forming a durable film with favorable attributes.

An undercoat according to the present invention would be comprised of the following ingredients.

A silicone elastomer such as NuLastic™ Silk-E D-99 LSA, (INCI: Isononyl Isononanoate, (and) Polysilicone 23) at 10-90% range combined with a water based thermoplastic resin such as Polyderm PPG-20, (INCI: POLYDERM PPG-20/IPDI Copolymer) at 10-90%. Preferable ranges are 40% of NuLastic™ Silk-E D-99 LSA+40% Polyderm PPG-20. Another embodiment uses 40% NuLastic™ Silk-E D-99 LSA+20% Polyderm PPG-20. These combinations and ratios are preferable but not exclusive. Colorants such as Iron Oxides, Titanium Dioxide, FD&C dyes and Lakes, can be used. Colorants as well as fillers such as mica, talc, etc. can be surface, treated or untreated. Esters, oils, solvents, Emulsifiers, waxes, preservative, and actives can be used to make a long lasting lip product.

The topcoat of a lipstick in two component long wear lip products can be made as a stick or as a lip gloss. Ingredients which are typically used are hydrogenated alkenes (polybutene or polydecene), esters, polyurethanes, oils (mineral, natural, petroleum) waxes, gellants, preservatives, pearlescents.

Examples of a gellant used in lipstick topcoats include the ingredients sold by Calumet Specialties Products and specifically under Calumet Penreco. The ingredients are sold under the trade name “Versagel”. Versagel M are specified for the personal care and cosmetic industry. These ingredients are using a Thermoplastic elastomer that gels non polar oils such as mineral oil, Polybutene, Isododecane, etc. One example of Calumet Penreco's gellant is: Versagel MN and the Inci is (Isononyl Isononanoate) Isononyl Isononanoate (and) Ethylene/Propylene/Styrene Copolymer (and) Butylene/Ethylene/Styrene Copolymer.

Gloss formulas:

| | |
|---|---|
| Hydrogenated Polybutene | 40% |
| Mineral Oil | 59.7% |
| Propyl Paraben | 0.3% |
| Versagel MN 750 | 50% |
| Hydrogented Polybutene | 49.7 |
| Preservative | 0.3% |
| Versagel MN 750 | 50% |
| Hydrogented Polybutene | 39.7 |
| Polyderm PPI-CO | 10% |
| Preservative | 0.3% |

### Stick Formulas

| | |
|---|---|
| Versagel MN 750 | 41% |
| Hydrogented Polybutene | 39.7 |
| Polyderm PPI-CO | 10% |
| Preservative | 0.3% |
| Polyethylene | 9% |

### Further Examples of Personal Care Compositions

### Transfer Resistant, Long Wear Lip Paint

| NuLastic ™ Silk E ID-LSA Isododecane, Polysilicone 23 | | 40% |
|---|---|---|
| Polyderm PPI PE/PA Polyether-Propionic Acid/TMXDI Copolymer | | 40% |
| Iron Oxides | | 4% |
| TiO2 | | 5% |
| Pearlescent | | 3% |
| Mica or Talc | | 2% |
| Wickenol 151 | Isononyl Isononanoate | 6% |

### Transfer Resistant, Water Resistant, Long Wear, Non Volatile Lip Paint, Highly Plasticized Film Formation

| NuLastic ™ Silk E D99-LSA (proposed) Isononyl Isononanoate, | | 40% |
|---|---|---|
| (and) Polysilicone 23 | | |
| Polyderm PPG-20 | (proposed) PPG-20/DMPA/IPDI Copolymer | 40% |
| Iron Oxides | | 4% |
| TiO2 | | 5% |
| Pearlescent | | 3% |
| Mica or Talc | | 2% |
| Dermol 99 | Isononyl Isononanoate | 6% |

### Transfer Resistant, Water Resistant, Long Wear, Non Volatile Lip Paint, Emulsion Stick

| NuLastic ™ Silk E D99-LSA (proposed) Isononyl Isononanoate, | | 17% |
|---|---|---|
| (and) Polysilicone 23 | | |
| Polyderm PPI-PE/PA | Polyurethane 18 | 20% |
| Isononyl Isononanoate | | 10% |
| Diisopropyl Dimer Dilinoleate | | 10% |
| Bis-PEG/PPG-14/14 Dimethicone | | 2% |
| Behenyl Alcohol | | 4% |
| Glyceryl Monostearate | | 3% |
| Polyethylene | | 9% |
| Water | | 20% |
| Iron Oxides | | 5% |

### Peel Off Mask

| NuLastic ™ Silk E D-99 LSA (proposed) Isononyl Isononanoate, | | 35% |
|---|---|---|
| | (and) Polysilicone 23 | |
| Polyderm | Polyether-Propionic Acid/TMXDI Copolymer | 35% |
| PPI-PE/PA | | |
| Clay | Illite | 29.5% |
| Preservative | | .5% |

### Transfer Resistant Eye Shadow

| | | |
|---|---|---|
| Wickenol 822 | Arachidyl Behenate | 5% |
| DC 1503 | Dimethicone, Dimethiconol | 3% |
| Abil Em-90 | Cetyl PEG/PPG-10/1-Dimethicone | 3% |
| Performalene 500 | Polyethylene | 4% |
| NuLastic ™ Silk E D-99 LSA | (proposed) Isononyl Isononanoate, | 30% |
| | (and) Polysilicone 23 | |
| Iron Oxides | | 5% |
| TiO2 | Titanium Dye | 4% |
| Pearlescent | | 6% |
| Polyderm PPG-20 | (proposed)PPG-20/DMPA/IPDI | 15% |
| | Copolymer | |

### Mascara

| NuLastic ™ Silk-E D-99 LSA (proposed) Isononyl Isononanoate, | | 15% |
|---|---|---|
| (and) Polysilicone 23 | | |
| Waxenol 822 | Arachidyl Behenate | 10% |
| Iron Oxide | | 10% |
| Water | | 49.0% |
| Polyderm PPI -PE/PA Polyether-Propionic Acid/TMXDI | | 15% |
| Copolymer | | |
| Preservative | | .5% |
| Xanthan Gum | | .5% |

### Two Part Mascara

Pigmented Base Coat

| | |
|---|---|
| NuLastic ™ SURFA D99-9 | 50% |
| Polyderm PPI-PE/PA | 30% |
| Black Iron Oxide | 10% |
| Isododecane | 5% |
| Wax | 2% |
| Isononyl Isononanoate | 3% |

Topcoat

| | | |
|---|---|---|
| Polyderm PPG-20 | (proposed)PPG-20/DMPA/IPDI Copolymer | 80% |

| | |
|---|---|
| NuLastic ™ Silk-E-ID-LSA | 20% |

### Skin Treatment Cream

| NuLastic ™ Silk-E D-99 LSA (propsed) Isononyl Isononanoate, | | 8% |
|---|---|---|
| (and) Polysilicone 23 | | |
| Wickenol 151 | Isononyl Isononanoate | 4% |
| DC 200 350 cst | Dimethicone | 3% |
| Water | | 80% |
| Sepinov EMT-10 Sodium Acrylate/Sodium | | 0.5% |
| Acryloyldimethyltaurate Copolymer | | |
| TEA 99% | Triethanolamine | 0.5% |
| Vitamin E | Tocopheryl Acetate | 1% |
| Preservative | | 0.5% |
| Aloe Vera | | 2.5% |

### Transfer Resistant, Long Wear Lip Paint

| NuLastic ™ Silk E ID-LSA Isododecane (and) Polysilicone 23 | | 40% |
|---|---|---|
| Polyderm PPI PE/PA Polyether-Propionic Acid/TMXDI Copolymer | | 30% |
| Iron Oxides | | 4% |
| TiO2 | | 5% |
| Pearlescent | | 3% |
| Mica or Talc | | 2% |
| Wickenol 151 | Isononyl Isononanoate | 6% |
| KP 545 | Isododecane, Silicone Acrylate | 10% |

### Transfer Resistant, Long Wear Lip Paint

| Shin Etsu's KSG 210 Dimethicone (and) Dimethicone/PEG-10/15 | | 40% |
|---|---|---|
| Crosspolymer | | |
| Polyderm PPI PE/PA Polyether-Propionic Acid/TMXDI Copolymer | | 30% |
| Iron Oxides | | 4% |
| TiO2 | | 5% |
| Pearlescent | | 3% |
| Mica or Talc | | 2% |
| Wickenol 151 | Isononyl Isononanoate | 6% |
| KP 545 | Isododecane, Silicone Acrylate | 10% |

### Further Examples

A number of polymer blends were prepared pursuant to the present invention using the following procedure and the characteristics of the films were assessed.

The following represents a generic manufacturing method used to produce compositions according to the present invention and resulting films, including final manufactured commercial products. These methods are applicable to all compositions according to the present invention and modifications to same may be made by those of skill in the art using standard well-known methods.

### Cold Process Method:

The first method is a cold process method. This method is the preferred method used to produce final compositions which contain no waxes or any other component which requires heat for mixing/formulation.
1. Place thermoset elastomer (TSE) in solvent into a vessel and begin mixing using a homogenizer with standard propeller blades;
2. Add thermoplastic elastomer/resin (TPE) in aqueous solvent to the vessel containing the TSE;
3. Mix 1 and 2 until homogeneous (about 1 hour)—external emulsifier, if required, may be added at this time to homogenize;
4. Add oil phase ingredients and mix until homogenous;
5. Add aqueous phase ingredients and mix until homogenous;
6. Add powders and other components (colorants, fillers, etc.);
7. Stir until completely mixed—Bulk is ready to fill.

### Second Method—Requiring Heat

1. Place TSE in solvent into a vessel and begin mixing using a homogenizer with standard propeller blades;
2. Add TPE/resin in aqueous solvent to the vessel containing the TSE;
3. Mix until homogeneous (about 1 hour)—note external emulsifier, if required, may be
   added at this time to homogenize;
4. Add aqueous phase ingredients and mix until homogenous;
5. Add oil phase ingredients (except waxes and other components which require heat to liquefy) and mix until homogenous;
6. Heat the vessel to 5° C. above the melting temperature of the highest melting ingredient;
7. Add powders such as colorants, fillers, etc., if needed;
8. Cool vessel to room temperature while mixing (if the bulk is too viscous at room temperature a higher temperature that allows the bulk to be moved is fine.
9. Bulk is ready to fill.

A number of blends were made according to the present invention (most excluded an external emulsifier) as described above. The results are presented in attached Table 1 (FIG. 1) hereof.

### Example

### Impact of Inclusion of Silicone Elastomer on Glass Transition of Thermoplastic Resin

Differential Scanning calorimetry (DSC) Analysis
Samples submitted for DSC analysis:

### Sample Polymer/Polymer Mixture

### JRM1-154, NuLastic™ Silk E ID LSA

### MB1-107-4, Polyderm PPI-PE/PA

### MB1-107-6, 50% NuLastic™ Silk E ID LSA+50% Polyderm PPI-PE/PA

### MB1-107-7, 50% NuLastic™ Silk E D99 LSA+50% Polyderm PPG-20

### MB1-107-8, 50% NuLastic™ Silk E D99 LSA+50% Polyderm PE/PA

Samples were mixed until homogenous. A spatula was used to spread an amount of material as indicated above onto a plastic substrate to provide a film. The substrate was placed in a 50 C. oven and allowed to cure overnight. The resulting film was detached from the plastic substrate. This film was submitted for DSC analysis.

### Results:

### JRM1-154, NuLastic™ Silk E ID LSA

Melt point at −42 C.

### MB1-107-4, Polyderm PPI-PE/PA

Glass transition at −42.82 C.

### MB1-107-6, 50% NuLastic™ Silk E ID LSA+50% Polyderm PPI-PE/PA

Glass transition at −64 C., Melt point at −2 C.

### MB1-107-7, 50% NuLastic™ Silk E D99 LSA+50% Polyderm PPG-20

Glass transition at −53 C., Melt point at 0 C.

### MB1-107-8, 50% NuLastic™ Silk E D99 LSA+50% Polyderm PE/PA

Glass Transition at −52 C., Melt point at −7 C.

### Conclusion:

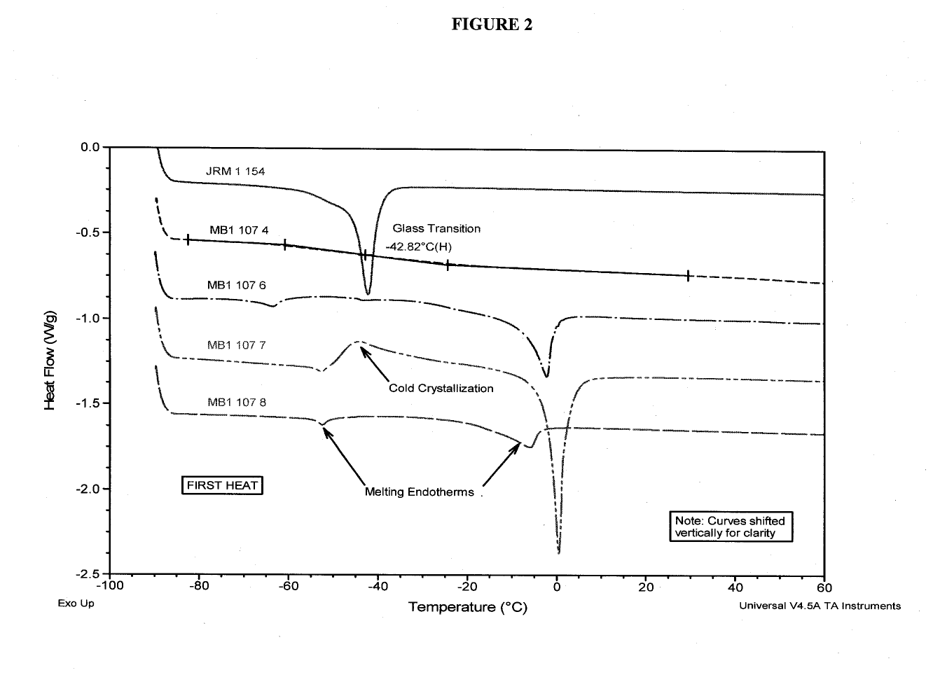

A) Glass transition is usually attributable to the TSE is shifting from −42 C. to −2, 0, and −7 C. when emulsified with a TPE.
   Plasticizing is defined as a softening of the film and is observed as a reduction in melt point.
   However, the inventors are observing the quite the opposite, something rather unexpected. The TSE's glass transition is increasing so therefore we can say that we are reinforcing the TSE when emulsified with a TPE. We have made the TSE harder or more crystalline.
   JRM1-154, Melt Point is −42 C.
   MB1-107-6 Melt point is −2 C., MB1-107-7 Melt Point is 0 C., MB1-107-8 Melt Point is −7 C.
B) Glass Transition of −42.82 C. is attributable to the TPE (thermoplastic resin). The TPE'S Glass Transition is decreasing form −42.82 C. to −64 C., −53 C., and −52 C. Plasticizing is defined as a softening of the film and is observed as a reduction in melt point.
   Thus, in the present invention, observed is a decrease in the glass transition of the emulsion blend. The TPE is actually being plasticized by the TSE.
C) This experiment actually quantifies analytically the proposed thesis that the TSE is plasticizing the TPE, resulting in a lowered TPE glass transition. The inventors have thus reduced the glass transition of the TPE and thus produced a film with superior characteristics.
   The inventors are quantifying an increase in film strength by the glass transition shift of the TSE. The films cast in some blends exhibit a paper like consistency as opposed to the plastic look of the TPE cast film.
   DSC Instrumental parameters.
   Samples were run on a TA instrument Q2000 DSC with Rcs-90 Cooling accessory. A heat-cool-reheat DSC method was used from −90 to 100 C. at 10 C./min.
   Samples were contained in Tzero Aluminum pans with lids.
   Sample size 12.5-13.0 mg was used.
   The results obtained are presented in attached FIG. 2, hereof

### Further Examples

### Compositions with Internal Plasticizer in Silicone Elastomer

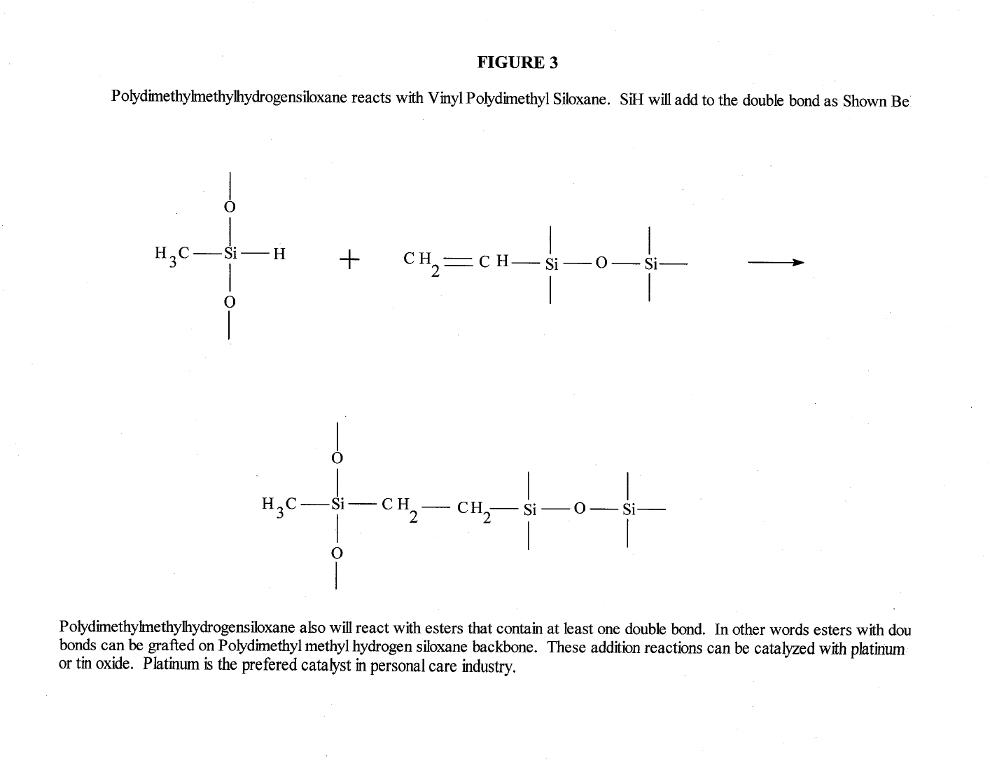

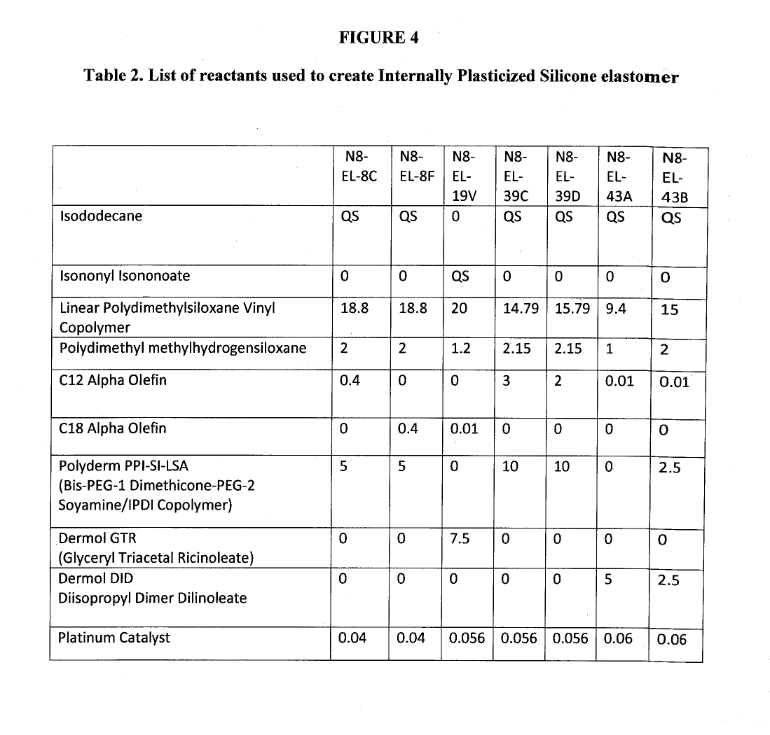

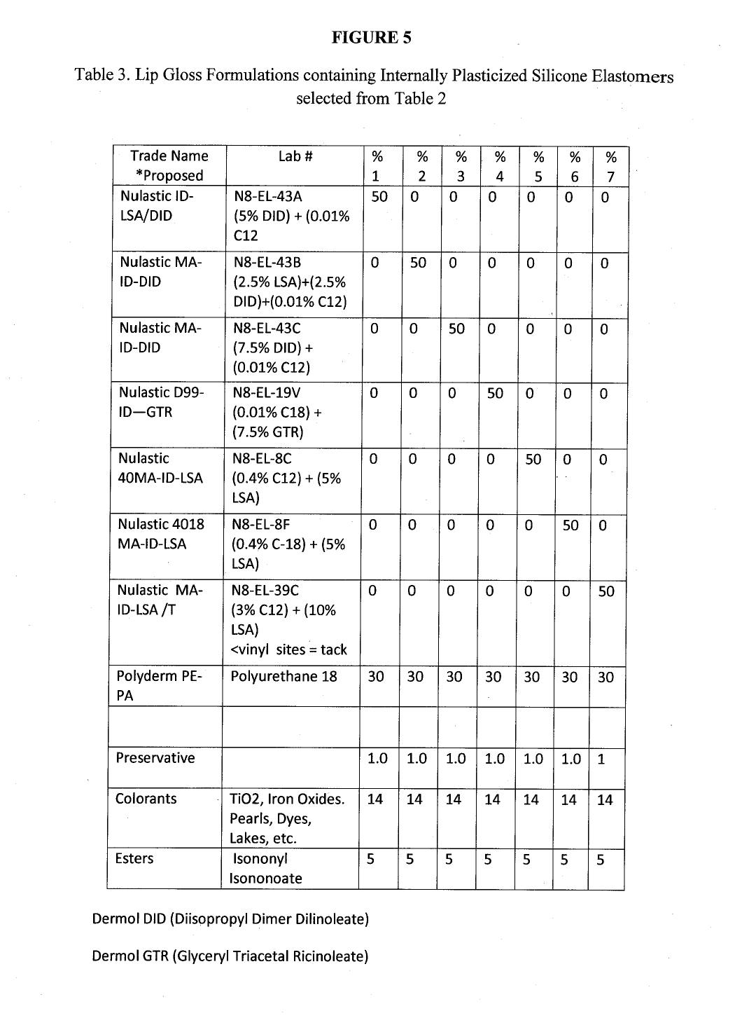

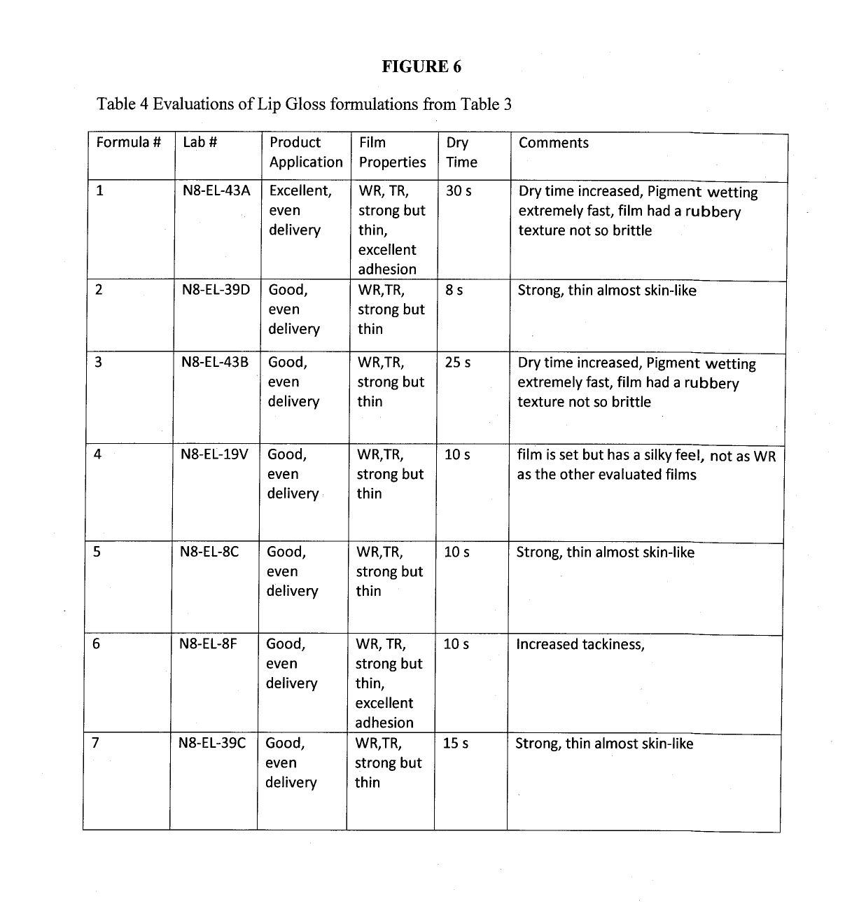

A number of silicone elastomers were synthesized using a minor modification of the reaction scheme which is presented in attached FIG. 3. In brief, the reaction occurs pursuant to the FIG. 3 reaction scheme, with the addition that a plasticizer ester which contains a double bond capable of reaction with Si—H groups on the silicone backbone pursuant to the description in FIG. 3 and otherwise as presented in the present application. In this way, plasticizer component can be covalently linked to the silicone elastomer, to increase the durability and wear resistance (among other attributes) of final compositions. Tables 2, 3 and 4 show the various compositions which are formulated from the silicone elastomer which contains covalently bound plasticizer. The results are presented below.

Conclusions: Internally Plasticized Silicone Elastomer when used in the present technology offer superior film formation. Superior to externally plasticized prototypes made with the present invention. The selection and use of these external esters would be limited due to compatibility issues with TPE's as well as the TSE are used in the present invention. This aspect of the invention (internal plasticizer content covalently linked to the silicone elastomer addresses these issues). Let's consider the issues when adding an external plasticizer to the TSE's claimed in the present invention. Due to the TSE's aversion to polar ingredients the use levels of polar ingredients is reduced and in some cases not possible at all. If we use Lab reference # N8-EL-43A (Table 1), internally plasticized with Diisopropyl Dimer Dilinoleate, as an example, we find that this TSE when used in the current invention exhibit some unexpected results. Formula #1 (Table 3) exhibited a longer dry time after the product was applied to the skin with a lip gloss applicator. This ester is a substantive ester so this is to be expected whether internally or externally plasticized. The unexpected result occurs in the ability to use smaller amounts in an internally plasticized TSE, with enhanced results. Thus, the use of an externally plasticized TSE can lead to stability issues over time, in contrast to an internal plasticizer which promotes stability.

Another unexpected result is the ability of N8-EL-43A to aid in the deagglomeration of colorants such as Iron Oxides, Tio2, etc. These colorants used can be surface treated or untreated. Dry colorants were used in all formulations listed in Table 3 and all were dispersed into the Blend of Silicone TSE+ Polyurethane TPE using a propeller mixer. No homogenizer was used to disperse colorants. Typical time period for dispersing colorants can range from 5-30 minutes (lab scale production 100 grams-1000 grams. When pigments were dispersed and the TSE used was N8-EL-43A we were able to disperse all colorants within 1-3 minutes.

The film attributes exhibited differences. Externally plasticized TSE's used in the current invention made films that were barely perceptible to the touch. Better diffusion of the film forming mix can lead to very thin but robust film formation. Better diffusion can be related to the surface tension of the film being applied to a surface. Viscosity, lower is better, also leads to better diffusion of the film former unto the substrate. Diffusion can also be defined as how efficiently the film former spreads and invades all or most of the substrate. This leads to an even thin film but with very good adhesive functionality.

N8-EL-19V (Table 1), exhibited surprising results as well. Formula 4 from table 3 reduced the water resistance of the film which was surprising. This could facilitate the removal of the product and could be a consumer benefit. The most surprising attribute this film offered is a sensorial attribute. This film when formed on the skin dried to the touch but had a very unique wet silkiness feel.
Internally plasticized silicone TSE's are offering up a novel approach to modifying not only the formation of the film but how it mixes with other ingredients. Either speeding up the dispersion of colorants or improving the solubility of polar ingredients. Formation of the film from an adhesive stand point has been improved. The films last up to 24 hours and eventually had to be removed. A film that lasts this long when using existing transfer resistant products in the market would produce a heavy feel on the lips. The films produced with internally plasticized TSE's are so thin that they are barely perceptible to the wearer.
Internally plasticized TSE's tend to be less problematic than an externally plasticized TSE. If the plasticizer is internalized it cannot migrate or exude out of the formed film. This leads to a better control over film attributes. The internalization of the plasticizer also leads to better solubility outcomes. Once internalized the solubility of the internally plasticized TSE needs to be considered as one unit which reduces complexity when mixing other ingredients into the present invention. If the plasticizer is externalized you now would have to consider the solubility parameters of the TSE and the solubility parameter of the plasticizer.

It is to be understood by those skilled in the art that the foregoing description and examples are merely illustrative of the present invention, and should in no way be interpreted as limiting the scope of the present invention. Variations of the detail presented herein may be made without departing from the spirit and scope of the present invention as defined by the following claims. All references made in the present application to publications and/or published patents/patent applications, are incorporated herein, to the extent relevant.

1. A polymeric composition comprising at least one thermoplastic resin dispersed in an aqueous solvent, preferably water or a mixture of water and alcohol; at least one elastomer in soluble, dispersible or gelled form in an elastomer solvent and an optional external emulsifier effective to emulsify said water and said solvent, wherein said thermoplastic resin comprises about 10% to about 80% by weight of said dispersion; said elastomer comprises about 10% to about 80% by weight of said elastomer and solvent, and wherein said emulsifier, when present, comprises about 0.01% to about 20% by weight of said thermoplastic resin, said elastomer, said aqueous solvent and said optional solvent in combination, said composition, when deposited onto a surface, dries (after evaporation of solvent and water) into a uniform, flexible, durable film exhibiting water resistance, flexibility, durability and optionally, transfer resistance (when the film also comprises a pigment, dye, oil and/or active), said thermoplastic resin comprising about 5% to about 95% of said film, said elastomer comprising about 1% to about 95% by weight of said film, and said external emulsifier, when present, comprising about 0.025% to about 10% by weight of said film.

2. The composition according to claim 1 wherein said elastomer is a thermoset silicone elastomer.

3. The composition according to claim 1 wherein said thermoset elastomer is a silicone thermoset elastomer which is gelled.

4. The composition according to claim 2 wherein said thermoset elastomer is chemically modified to contain internal plasticizers.

5. The composition according to claim 2 wherein said thermoplastic resin is selected from the group consisting of acrylonitrile butadiene styrene polymers (ABS), polyacrylic or poly(meth)acrylic resins (PMMA), celluloid cellulose acetate, cyclic olefin copolymers (COC), ethylene-vinyl acetate (EVA), ethylene vinyl alcohol (EVOH), fluoroplastics, acrylic/PVC copolymer, liquid crystal polymer (LCP), polyacrylonitrile (PAN or acrylonitrile), polyoxymethylene (POM), polyamide (nylon), polycarbonate, polyamide-imide (PAD, polyaryletherketone (PAEK), polybutadiene (PBD), polybutadiene/styrene copolymers, polybutadiene/acrylic copolymers, polybutadiene/acrylamide copolymers, polybutylene (PB), polybutylene terephthalate (PBT), polycaprolactone (PCL), polychlorotrifluoroethylene (PCTFE), polyethylene terephthalate (PET), polyhydroxyalkanoates (PHAs), polyketone (PK), polyester, polyethylene (PE, both low and high density), polyetheretherketone (PEEK), polyetherketoneketone (PEKK), polyaryletherketone (PAEK), polyetherimide (PEI), polyethersulfone (PES), chlorinated polyethylene (CPE), polyimide (PI), polylactic acid (PLA), polymethylpentene (PMP), polyphenylene, polyphenylene oxide (PPO), polyphenylene sulfide (PPS), polyphthalamide (PPA), polypropylene (PP), polystryrene (PS), polysulfone (PSU), polytrimethylene terephthalate (PTT), polyurethane (PU), polyvinyl acetate (PVA), polyvinyl chloride (PVC), polyvinylidene chloride (PVDC) and styrene-acrylonitrile, among others.

6. The composition according to claim 1 wherein said elastomer is a crosslinked hydrophilic silicone thermoset elastomer.

7. The composition according to claim 1 wherein said elastomer is a silicone hydrocarbon crosslinked elastomer.

8. The composition according to claim 7 wherein said silicone hydrocarbon crosslinked elastomer is the reaction product of polybutadiene or a multi-unsaturated polyurethane which is crosslinked (or chain-extended) with a bis-hydrosilane terminated polysiloxane.

9. The composition according to claim 1 wherein said aqueous solvent is water or a mixture of water and an alcohol.

10. (canceled)

11. (canceled)

12. The composition according to claim 1 wherein said elastomer solvent is a ketone, isododecane, an ester, isohexadecane, dimethicone and cyclomethicone.

13. The composition according to claim 1 wherein said composition further comprises an external plasticizer.

14. The composition according to claim 1 wherein said elastomer is a plasticizer of said thermoplastic resin.

15. The composition according to claim 1 further comprising an external emulsifier.

16. The composition according to claim 1 wherein said thermoplastic resin and/or said elastomer is a self-emulsifier.

17. The composition according to claim 1 comprising at least one water based thermoplastic resin, at least one silicone elastomer, at least one volatile or non-volatile solvent, water, water based actives, preservatives, colorants, oils and esters.

18. The composition according to any of claims 1-17 claim 1 formulated for use on keratinous surfaces.

19. The composition according to claim 18 wherein said keratinous surfaces are skin, hair, nails or lips.

20. The composition according to claim 1 formulated for use on non-keratinous surfaces.

21. The composition according to claim 20 wherein non-keratinous surfaces are glass, leather, wood, plastic or rubber.

22. The composition according to claim 1 wherein said thermoplastic resin is PPG-20/DMPA/IPDI (polyurethane-36) copolymer.

23. The composition according to claim 1 wherein said thermoplastic resin is polyether-propionic acid/TMXDI copolyimer (polyurethane-18)

24. The composition according to claim 1 wherein the elastomer is Polysilicone 23.

25. The composition according to claim 1, wherein the thermoplastic resin is present in amount ranging from about 5%-80% by weight of said film.

26. (canceled)

27. (canceled)

28. (canceled)

29. (canceled)

30. The composition of claim 24, wherein the silicone elastomer is Polysilicone 23 and is present at about 1% to about 30% by weight of a solvent.

31. The composition according to claim 1 wherein said thermoplastic resin is present at about 40% by weight of said film and said silicone elastomer is present at about 40% by weight of said film.

32. The composition according to claim 1 wherein said thermoplastic resin is present at about 25% by weight of said film and said silicone elastomer is present at about 40% by weight of said film.

33. The composition according to claim 1 wherein said film is continuous.

34. The composition according to claim 1 wherein said film exhibits the film properties of transfer resistance, water resistance and durability.

35. The composition according to claim 1 wherein said elastomer solvent is non-volatile.

36. The composition according to claim 1 wherein said elastomer solvent is volatile.

37. A polymeric composition comprising at least one thermoplastic resin dispersed in an aqueous solvent, preferably water or a mixture of water and alcohol; at least one elastomer in soluble, dispersible or gelled form in an elastomer solvent wherein said elastomer is chemically modified to contain at least one internal plasticizer, and an optional external emulsifier effective to emulsify said water and said solvent, wherein said thermoplastic resin comprises about 10% to about 80% by weight of said dispersion; said elastomer comprises about 10% to about 80% by weight of said elastomer and solvent, and wherein said emulsifier, when present, comprises about 0.01% to about 20% by weight of said thermoplastic resin, said elastomer, said aqueous solvent and said solvent in combination, said composition, when deposited onto a surface, dries (after evaporation of solvent and water) into a uniform, flexible, durable film exhibiting water resistance, flexibility, durability and optionally, transfer resistance (when the film also comprises a pigment, dye, oil and/or active), said thermoplastic resin comprising about 5% to about 95% of said film, said elastomer comprising about 1% to about 95% by weight of said film, and said external emulsifier, when present, comprising about 0.025% to about 10% by weight of said film.

38. (canceled)

39. The composition according to claim 37 wherein said elastomer is a thermoset silicone elastomer which is formed by reacting an activated polysiloxane polymer with a plasticizer compound to form a plasticizer-containing polysiloxane polymer containing a plurality of plasticizer compounds covalently bonded to said polysiloxane polymer.

40. (canceled)

41. (canceled)

42. The composition according to claim 41 wherein said polysiloxane polymer may be crosslinked with polysiloxane polymers which contain carbon-carbon double bonds, thus forming a gelled thermoset silicone elastomer.

43. The composition according to claim 39 wherein said activated polysiloxane polymer contains a plurality of Si—H groups reactive with a plasticizer compound containing vinyl groups (carbon-carbon double bonds) wherein said polysiloxane polymer forms a covalent Si—C bond with said plasticizer compound.

44. The composition according to claim 39 wherein said plasticizer compound is selected from the group consisting of esters that contain double bonds and can be grafted onto activated polysiloxane polymers at Si—H groups, esters that contain or can be reacted to contain double bonds, alcohols that contain or can be reacted to contain double bonds, or mixtures thereof.

45. The composition according to claim 44 wherein said plasticizer compound is cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, Di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂-C₁₃ alkyl lactate, C₁₂-C₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂-C₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxstearate or a mixture thereof.

46. The composition according to claim 37 wherein said elastomer is a thermoset silicone elastomer which is formed by reacting a polysiloxane polymer which contains between 4 and 25 Si—H groups which are reactive with a vinyl group in a plasticizer compound wherein said Si—H groups and said reactive double bond in said plasticizer compound form a Si—C bond covalently linking said silicone elastomer to said plasticizer compound.

47. The composition according to claim 46 wherein said plasticizer compound is selected from the group consisting of esters that contain double bonds and can be grafted onto activated polysiloxane polymers at Si—H groups, esters that contain or can be reacted to contain double bonds, alcohols that contain or can be reacted to contain double bonds, or mixtures thereof.

48. The composition according to claim 47 wherein said plasticizer compound is cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, Di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂-C₁₃ alkyl lactate, C₁₂-C₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂-C₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxstearate or a mixture thereof.

49. The composition according to claim 39 wherein said activated siloxane polymer is according to the chemical structure: 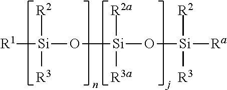
Where R¹ and R^{a} are independently H, an optionally substituted C₁-C₆ alkyl group or an optionally substituted C₂-C₆ alkenyl group;
Each R² and R³ is independently H, OH, or a C₁-C₃ alkyl group;
Each R^{2a} and R^{3a} is independently H, OH, or a C₁-C₃ alkyl group,
n is from 5 to 50,000, and
j is from 0 to 50, wherein said activated siloxane polymer contains about 4 to about 25 Si—H groups.

50. The composition according to claim 49 wherein said activated siloxane polymer is reacted with a plasticizer compound selected from the group consisting of esters that contain double bonds and can be grafted onto activated polysiloxane polymers at Si—H groups, esters that contain or can be reacted to contain double bonds, alcohols that contain or can be reacted to contain double bonds, or mixtures thereof.

51. The composition according to claim 50 wherein said plasticizer compound is cetyl ricinoleate, diisopropyl dimer dilinoleate, decyl oleate, glyceryl monooleate, isostearyl erucate, methyl acetyl ricinoleate, oleyl erucate, oleyl lactate, oleyl oleate, propylene glycol ricinoleate, arachidyl propionate, arachidyl behenate, dicapryl maleate, Di-C₁₂₋₁₅ alkyl fumarate, linoleamidopropyl ethyldimonium ethosulphate, glyceryl triacetyl ricinoleate, glyceryl diricinoleate, glyceryl diricinoleate copolymer, octyldodecyl hydroxystearate, C₁₂-C₁₃ alkyl lactate, C₁₂-C₁₅ alkyl lactate, cetyl lactate, ethoxydiglycol, glycereth-7 citrate, glycereth-7 lactate, isocetyl salicylate, isodecyl salicylate, isodecyl oleate, isopropyl myristate, isostearyl lactate, glycereth 4.5 lactate, lauryl lactate, myristyl lactate, C₁₂-C₁₅ alkyl salicylate, propylene glycol benzoate, propylene glycol lactate, tridecyl salicylate, glycerol-7 hydroxystearate, ethylene glycol distearate, glyceryl hydroxystearate, glyceryl stearate, propylene glycol stearate, tricapryl citrate, triisocetyl citrate, trioctyldodecyl citrate, isostearyl stearoyl stearate, glyceryl triacetyl hydroxstearate or a mixture thereof.

52. A personal care composition comprising a composition according to claim 1 further in combination with at least one additional component selected from the group consisting of water, an aqueous solvent, a non-aqueous solvent, emollients, humectants, oils, conditioning agents, emulsifiers, surfactants, thickeners, stiffening agents, medicaments, fragrances, preservatives, deodorant components, anti-perspirant compounds, skin protecting agents, pigments, sunscreens and mixtures thereof.

53. (canceled)

54. A pharmaceutical composition formulated for topical or transdermal delivery into a patient comprising a composition according to claim 1 further in combination with an effective amount of at least one bioactive agent in combination with a pharmaceutically acceptable carrier, additive or excipient.

55. (canceled)

56. (canceled)

57. A personal care composition comprising at least one thermoplastic resin dispersed in an aqueous solvent; at least one elastomer in soluble, dispersible or gelled form in a solvent wherein said elastomer is optionally chemically modified to contain at least internal plasticizer, and an optional external emulsifier effective to emulsify said water and said solvent, wherein said thermoplastic resin comprises about 10% to about 80% by weight of said dispersion; said elastomer comprises about 10% to about 80% by weight of said elastomer and solvent, and wherein said emulsifier, when present, comprises about 0.01% to about 20% by weight of said thermoplastic resin, said aqueous solvent. said elastomer and said elastomer solvent in combination, said composition further comprising at least one additional component selected from the group consisting of water, an aqueous solvent, a non-aqueous solvent, emollients, humectants, oils, conditioning agents, emulsifiers, surfactants, thickeners, stiffening agents, medicaments, fragrances, preservatives, deodorant components, anti-perspirant compounds, skin protecting agents, pigments, sunscreens and mixtures thereof, wherein said composition, when deposited onto a surface, dries into a uniform, flexible, durable film exhibiting water resistance, flexibility, durability and optionally, transfer resistance when the film also comprises a pigment, dye, oil and/or active, said thermoplastic resin comprising about 5% to about 95% of said film, said elastomer comprising about 1% to about 95% by weight of said film, and said external emulsifier, when present, comprising about 0.025% to about 10% by weight of said film.

58. (canceled)

59. (canceled)

60. A two part composition the composition comprising a part A mixture and a part B mixture,
the part A mixture comprising at least one thermoplastic resin dispersed in an aqueous solvent and additional optional additives or bioactive agents;
the part B mixture comprising at least one elastomer in soluble, dispersible or gelled form in a solvent wherein said elastomer is optionally chemically modified to contain at least one internal plasticizer, said part A mixture and/or said part B mixture comprising an optional external emulsifier effective to emulsify said aqueous solvent and said elastomer solvent when said part A mixture and said part B mixture are combined, wherein said thermoplastic resin comprises about 10% to about 80% by weight of said dispersion; said elastomer comprises about 10% to about 80% by weight of said elastomer and solvent and wherein said emulsifier, when present, comprises about 0.01% to about 10% by weight of said thermoplastic resin, said aqueous solvent, said elastomer and said solvent, said composition, when deposited onto a surface, dries into a uniform, flexible, durable film exhibiting water resistance, flexibility, durability and optionally, transfer resistance when the film also comprises a pigment, dye, oil and/or active, said thermoplastic resin comprising about 5% to about 95% of said film, said elastomer comprising about 1% to about 95% by weight of said film, and said external emulsifier, when present, comprising about 0.025% to about 10% by weight of said film.

61. The composition according to claim 60 wherein said part A mixture and/or said part B mixture comprise an additional component selected from the group consisting of water, an aqueous solvent, a non-aqueous solvent, emollients, humectants, oils, conditioning agents, emulsifiers, surfactants, thickeners, stiffening agents, medicaments, fragrances, preservatives, deodorant components, anti-perspirant compounds, skin protecting agents, pigments, sunscreens and mixtures thereof.

62. The composition according to claim 61 wherein said additional component(s) comprise about 0.1% to about 80% by weight of the final film.

63. (canceled)

64. (canceled)

65. (canceled)

66. (canceled)

67. (canceled)

68. (canceled)

69. (canceled)

70. (canceled)

71. The composition according to claim 61 wherein said elastomer is a crosslinked hydrophilic silicone thermoset elastomer.

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. The composition according to claim 61 wherein said thermoplastic resin is PPG-20/DMPA/IPDIcopolymer or polyether-propionic acid/TMXDI copolymer.

85. (canceled)

86. The composition according to claim 61 wherein the elastomer is Polysilicone 23.

87. (canceled)

88. (canceled)

89. (canceled)

90. (canceled)

91. (canceled)

92. (canceled)

93. (canceled)

94. The composition according to claim 61 wherein said film is continuous.

95. (canceled)

96. (canceled)

97. (canceled)

98. (canceled)

99. (canceled)

100. A method of plasticizing a thermoplastic resin, said method comprising combining with at least one thermoplastic resin dispersed in an aqueous solvent, at least one elastomer in soluble, dispersible or gelled form in an elastomer solvent wherein said elastomer is optionally chemically modified to contain at least one internal secondary plasticizer and an optional external emulsifier effective to emulsify said water and said solvent, wherein said thermoplastic resin comprises about 10% to about 80% by weight of said dispersion; said elastomer comprises about 10% to about 80% by weight of said elastomer and solvent, and wherein said emulsifier, when present, comprises about 0.01% to about 20% by weight of said thermoplastic resin, said elastomer, said aqueous solvent and said solvent in combination, said composition, when deposited onto a surface, dries (after evaporation of solvent and water) into a uniform, flexible, durable film exhibiting water resistance, flexibility, durability and optionally, transfer resistance (when the film also comprises a pigment, dye, oil and/or active), said thermoplastic resin comprising about 5% to about 95% of said film, said elastomer comprising about 1% to about 95% by weight of said film, and said external emulsifier, when present, comprising about 0.025% to about 10% by weight of said film.

101. (canceled)

102. (canceled)

103. (canceled)

104.-116. (canceled)
